(19) United States
(12) Patent Application Publication
(10) Pub. No.: US 2025/0298027 A1
(43) Pub. Date: Sep. 25, 2025

(54) METHOD OF EVALUATING SMALL MOLECULE DISTRIBUTION USING TELLUROPHENE ANALOGUES

(71) Applicants: THE GOVERNING COUNCIL OF THE UNIVERSITY OF TORONTO, Toronto (CA); UNIVERSITY HEALTH NETWORK, Toronto (CA)

(72) Inventors: Mark Nitz, Markham (CA); Rahul Rana, Markham (CA); Bradly Wouters, Markham (CA); Ravi Vellanki, Markham (CA); Nicole Dawn Potter, Markham (CA)

(21) Appl. No.: 18/860,236

(22) PCT Filed: Apr. 28, 2023
(86) PCT No.: PCT/IB2023/000247

§ 371 Date: Oct. 25, 2024

Related U.S. Application Data
(60) Provisional application No. 63/335,926, filed on Apr. 28, 2022.

(51) Int. Cl. G01N 33/68; C07K 7/06; G01N 27/62; G01N 33/50

(52) U.S. Cl. CPC G01N 33/68; C07K 7/06; G01N 27/62; G01N 33/5005; C07B 2200/05; G01N 2458/15

(57) ABSTRACT

The present disclosure relates to tellurophene analogues of small molecules, and methods of detecting small molecules using their tellurophene analogues. The present disclosure further relates to compositions and kits comprising tellurophene analogues of the present disclosure. The present disclosure also relates to methods of determining a dosage amount of a small molecule.

### RELATED APPLICATIONS

This application claims priority to U.S. provisional application No. 63/335,926, filed on Apr. 28, 2022, entitled “Method of Evaluating Small Molecule Distribution Using Tellurophene Analogues,” which is incorporated herein by reference in its entirety.

### FIELD

The present disclosure relates to tellurophene containing small molecule analogues and methods of detecting a small molecule within a sample obtained from a subject or a cell/tissue culture using the tellurophene as a reporter. The present disclosure also relates to a tellurophene teniposide analogue and a tellurophene carfilzomib analogue. The present disclosure further includes uses of the analogues of the present disclosure in the evaluation of a distribution of a small molecule. Moreover, the present disclosure relates to methods of determining a dosage amount of a small molecule.

### INTRODUCTION

The biodistribution of exogenously administered small molecule therapeutics is rarely homogenous in vivo. Yet, target engagement at the desired location is key to translating a promising in vitro result into an in vivo response. Currently, methods to monitor biodistribution and target engagement have largely focused on assays carried out in bulk, and few methods give cellular resolution, especially in whole organismal models. Early approaches to monitor cellular biodistribution of small reversible inhibitors used microautoradiography, but due to the high specific activities required, the long exposure times and the technical challenges, this method has not been widely adopted. While advances in fluorescence-based tissue histological techniques have enabled the visualization and quantification of target engagement in processed tissue samples, these assays are limited in their ability to simultaneously identify and characterize the phenotypes associated with the cell types present. Due to the inherent challenges with multiplexing fluorescence-based microscopy, combining biodistribution with cellular characterization is best suited to IMAGING MASS CYTOMETRY™ (IMC™).

IMC™ is a high-dimensional tissue imaging platform that builds on CYTOF® mass cytometry (MC) technology, and is designed for epitope measurements on tissue sections. Earlier versions of the platform were introduced by the Bodenmiller group in 2014 where a panel of 32 antibodies was used to examine underlying tumour heterogeneity in human breast cancer samples. More recent examples have interrogated over 40 markers that can be quantified with subcellular resolution using current IMC™ reagents, and workflows, thus enabling deep profiling of individual cells in their native microenvironments. Spatially resolved information acquired by IMC™ can be analyzed to study cellular phenotypes in relation to spatial organization. Furthermore, with libraries of the appropriate probes, temporal changes in the tissue can be followed as has been done with hypoxia. Overlaying this information with small molecule therapeutic biodistribution would facilitate the design of dosing regimes and assist in understanding the perturbed cellular biochemistry.

Despite this progress, the potential for IMC™ has been underexplored in drug discovery and pharmaceutical development. To follow localization of a drug in tissues with IMC™, the molecule must bear a heavy isotope (>80 amu) compatible with the mass cytometer. Qing et al. exploited the ability of IMC™ to detect platinum isotopes arising from dosing the chemotherapeutic cisplatin, allowing visualization of the biodistribution in pancreatic cancer patient-derived xenografts. This landmark study revealed unexpected drug distribution as extensive unexpected binding of platinum to collagen fibers in the tumour stroma and normal tissues was observed. These findings provide insight into the long clearance times of cisplatin and some of the observations about the long-term toxicity of the therapy. While this work demonstrated the applicability of IMC™ to follow localization of cisplatin, few therapeutic agents have a MC-compatible element present to allow their detection.

Accordingly, there exists a need to develop a method to allow for application of mass cytometry to small molecules in general without requiring a MC-compatible element.

### SUMMARY

Any of the embodiments disclosed herein can be used in combination with one or more of the other embodiments.

It has been shown that the detection of biologically active small molecules by IMC™ beyond metallodrugs is possible using IMC-visible tellurophene analogues of the small molecules. It has also been shown that the analogues can be accessed by isosteric substitution of a five- or six-membered aromatic ring with a tellurophene. Tellurophenes are stable, biocompatible mass tags for MC and IMC™. As examples, this bioisostere design strategy has been successfully implemented to develop tellurophene-teniposide analogue and tellurophene-carfilzomib analogues. The exemplary analogues retained similar biological activity compared to the original small molecule and were detectable and quantifiable by MC.

Accordingly, in one aspect, the present disclosure includes a method of detecting of a small molecule within a sample, the method comprising
providing a sample obtained from a subject or from a cell/tissue culture that has been administered a tellurophene small molecule analogue comprising a tellurophene, the tellurophene comprising a tellurium atom; and
performing mass spectrometry on the sample to determine a level of the tellurium atom;
   wherein the small molecule has a structure comprising one or more monocyclic or bicyclic aromatic rings, and the analogue has a structure where at least one of the one or more aromatic rings of the structure of the small molecule is replaced with the tellurophene, and wherein the level of the tellurium atom corresponds to the level of the analogue and detection of the analogue is indicative of detection of the small molecule in the sample.

In another aspect, the present disclosure includes a use of a tellurophene analogue in the detection of a small molecule by mass spectrometry in a subject that has been administered the analogue or a cell/tissue culture that has been administered the analogue, wherein the small molecule has a structure comprising one or more monocyclic or bicyclic aromatic rings, and the analogue has a structure where at least one of the one or more monocyclic or bicyclic aromatic rings of the structure of the small molecule is replaced with a tellurophene.

In another aspect, the present disclosure includes a compound of Formula I

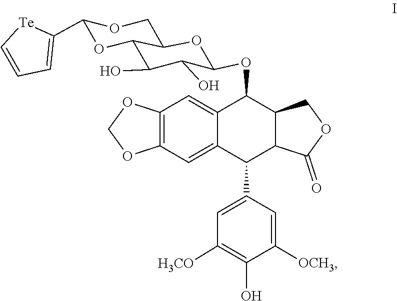

or a salt or solvate thereof.

In another aspect, the present disclosure includes a compound of Formula II

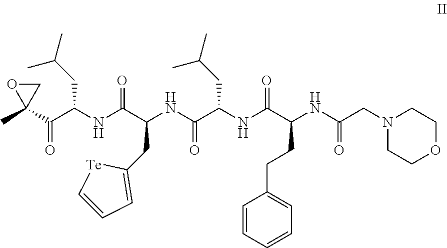

or a salt or solvate thereof.

In another aspect, the present disclosure includes a composition comprising the compound of the present disclosure and a carrier or excipient.

In another aspect, the present disclosure includes a use of the compound of Formula I, or the composition comprising the compound of Formula I of the present disclosure in the detection of teniposide by mass spectrometry in a subject that has been administered the compound of Formula I or salt or solvate thereof or a cell/tissue culture that has been administered the compound of Formula I or salt or solvate thereof.

In another aspect, the present disclosure includes a use of the compound of Formula II, or the composition comprising the compound of Formula II of the present disclosure in the detection of carfilzomib by mass spectrometry in a subject that has been administered the compound of Formula II or salt or solvate thereof or a cell/tissue culture that has been administered the compound of Formula II or salt or solvate thereof.

In another aspect, the present disclosure includes a kit for mass cytometry analysis comprising a tellurophene analogue of teniposide of Formula I

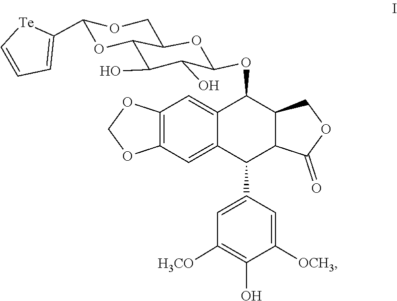

or a salt or solvate thereof, and
a tellurium standard or a plurality of tellurium standards.

In another aspect, the present disclosure includes a kit for mass spectrometry analysis, optionally mass cytometry analysis comprising a tellurophene analogue of carfilzomib of Formula II

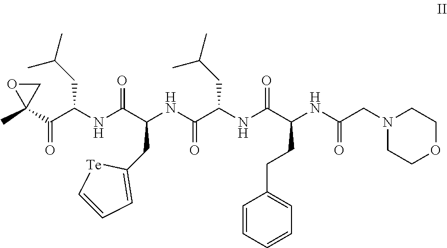

or a salt or solvate thereof, and
a tellurium standard or a plurality of tellurium standards.

In another aspect, the present disclosure includes a method of determining a dosage amount of a small molecule to achieve a desired target engagement of the small molecule in a subject or a cell/tissue culture, wherein the small molecule engages a target in the subject or the cell/tissue culture and produces a measurable effect at the target, the method comprising
detecting the small molecule in a subject by a method of the present disclosure;
measuring the effect produced by the small molecule; and
determining a dosage amount of the tellurophene analogue suitable to achieve the desired distribution of the small molecule, wherein the dosage amount of the tellurophene analogue indicates the dosage amount of the small molecule.

In one aspect a method a method of detecting a small molecule compound within a sample, the method comprising the steps of providing either a subject or a cell/tissue culture specimen, providing a small molecule analogue of a small molecule compound, wherein the small molecule compound has a structure comprising one or more monocyclic or bicyclic aromatic rings, and the small molecule analogue has a structure where at least one of the one or more monocyclic or bicyclic aromatic rings of the structure of the small molecule compound is replaced with tellurophene comprising a tellurium atom, thereby forming a tellurophene small molecule analogue, administering the tellurophene small molecule analogue to the subject or cell/tissue culture specimen, providing a sample which is taken either from the subject or from the cell/tissue culture specimen after administration of the tellurophene small molecule analogue, performing mass spectrometry on the sample to determine a level of the tellurium atom present in the sample, wherein the level of the tellurium atom corresponds to the level of the tellurophene small molecule analogue, and detection of the tellurophene small molecule analogue is indicative of detection of the small molecule compound in the sample.

In various embodiments, such as those described above, the method further includes quantifying the amount of the tellurium atom in the sample, wherein the tellurium atom level is indicative of the small molecule compound quantity in the sample. In various embodiments, such as those described above, the method further comprises quantifying one or more other analytes within the sample, the method further comprising labelling the sample with one or more mass tagged analyte binders prior to performing mass spectrometry, and determining a level of the one or more analyte binders. In various embodiments, such as those discussed above, the one or more mass tagged analyte binders are selected from the group consisting of metal-labelled antibodies, optionally a polymer-labelled antibodies, metal-labelled oligonucleotides, polymer-labelled oligonucleotides, intercalators such as 5-iodo-2′-deoxyuridine (IdU), and metal-containing intercalators (e.g. Rh-containing intercalator, and Ir-containing intercalator), metal-containing viability indicator such as cisplatin, barcoding reagents such as Cd-labelled CD45 and Pt-labelled CD45, and combinations thereof. In various embodiments, such as those described above, the subject is a mammal, optionally a mouse, a rat or a human.

In various embodiments, such as those described above, the mass spectrometry is performed at a plurality of discrete locations and the level of the tellurium atom is determined at each of the plurality of discrete locations to provide a distribution of the small molecule analogue within the sample, and wherein the distribution of the analogue within the sample is indicative of a distribution of the small molecule compound within the sample. In various embodiments, such as those discussed above, the sample is a single cell and the distribution of the small molecule analogue within the single cell is indicative of a distribution of the small molecule compound at a subcellular level. In various embodiments, such as those discussed above, a plurality of samples is provided and the samples comprise different tissues, cells or secretions of the subject, and wherein the detection of the small molecule analogue within the plurality of samples provides a distribution of the small molecule analogue within the subject which is indicative of a distribution of the small molecule compound within the subject, such as tissue or organ distribution. In various embodiments, such as those discussed above, the sample is or comprises urine, stool, blood or a fraction thereof, cerebrospinal fluid (CSF), saliva, muscle cell, fat cell, bone cell, hair, nail, skin cell, tumour cell or secretions, liver cell or secretions, heart cell, lung cell or secretions, pancreas cell or secretions, and/or stomach cell or secretions. In various embodiments, such as those discussed above, the sample is a frozen tissue section. In various embodiments, such as those discussed above, the sample is or comprises a cell from the cell culture or culture media from the cell culture. In various embodiments, such as those discussed above, the sample is a single cell and the distribution of the analogue within the single cell is indicative of a distribution of the small molecule compound at a subcellular level. In various embodiments, such as those discussed above, the mass spectrometry is mass cytometry or multiplex ion beam imaging, optionally the mass cytometry is mass cytometry imaging or mass cytometry suspension. In various embodiments, such as those discussed above, the tellurium atom is selected from ¹²⁰Te, ¹²²Te, ¹²³Te, ¹²⁴Te, ¹²⁵Te, ¹²⁶Te, ¹²⁸Te, ¹³⁰Te, and combinations thereof. In various embodiments, such as those discussed above, the tellurium atom comprises a plurality of tellurium isotopes and the mass cytometry is multichannel mass spectrometry, optionally multichannel mass cytometry. In various embodiments, such as those discussed above, the one or more monocyclic aromatic rings are 5- or 6-membered aromatic ring, optionally the one or more monocyclic 5- or 6-membered aromatic rings are independently selected from thiophene, furan, pyrrole, phenyl, or pyridine. In various embodiments, such as those discussed above, the one or more bicyclic aromatic rings are independently selected from naphthyl, indole, benzothiophene, or benzofuran.

In various embodiments, such as those discussed above, the structure of the small molecule compound comprises one or more monocyclic 5- or 6-membered aromatic rings and at least one of the monocyclic 5- or 6-membered aromatic rings is replaced with the tellurophene. In various embodiments, such as those discussed above, the structure of the small molecule compound comprises one or more bicyclic aromatic rings, and the at least one of the bicyclic aromatic rings is replaced with a benzo[b]tellurophene or a 4H-telluropheno[3,2-b]pyrrole. In various embodiments, such as those discussed above, the structure of the small molecule compound comprises one or more 5-membered aromatic rings, optionally the one or more 5-membered aromatic rings are each independently selected from thiophene, furan, or pyrrole. In various embodiments, such as those discussed above, the one or more 5-membered aromatic rings are thiophene. In various embodiments, such as those discussed above, the small molecule compound is teniposide and the tellurophene small molecule analogue is as shown in Formula I

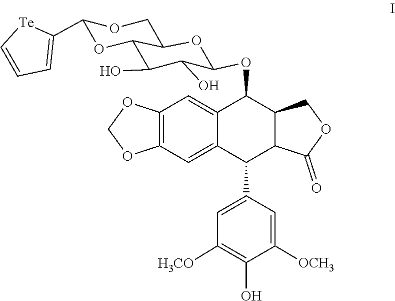

or a salt or solvate thereof.

In various embodiments, such as those discussed above, the structure of the small molecule compound comprises one or more 6-membered aromatic rings, optionally the one or more 6-membered aromatic rings are each independently selected from phenyl or pyridine, optionally the one or more 6-membered aromatic rings are phenyl. In various embodiments, such as those discussed above, the small molecule compound is carfilzomib and the tellurophene small molecule analogue is as shown in Formula II

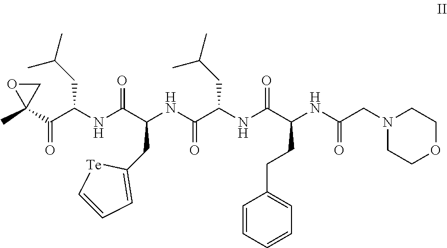

or a salt or solvate thereof.

In various embodiments, such as those discussed above, the small molecule analogue interacts irreversibly, optionally covalently, with a target in the sample. In various embodiments, such as those discussed above, the subject or the cell/tissue culture specimen had been administered the tellurophene small molecule analogue in combination with the small molecule compound, wherein the method further comprises detecting directly a level of the small molecule compound, and wherein a comparison of the level of the small molecule compound and the level of the analogue is indicative of a putative competitive binding of the analogue.

In another aspect, a use of a tellurophene small molecule analogue in a method of detection of a small molecule compound in a sample comprising the steps of providing either a subject or cell/tissue culture specimen, providing a small molecule analogue of a small molecule compound, wherein the small molecule compound has a structure comprising one or more monocyclic or bicyclic aromatic rings, and the small molecule analogue has a structure where at least one of the one or more aromatic rings of the structure of the small molecule compound is replaced with tellurophene comprising a tellurium atom, thereby forming a tellurophene small molecule analogue, administering the tellurophene small molecule analogue to the subject or cell/tissue culture specimen, providing a sample which is either taken from the subject or from the cell/tissue culture specimen after administration of the tellurophene small molecule analogue, performing mass spectrometry on the sample to determine a level of the tellurium atom present in the tellurophene small molecule analogue. In various embodiments, such as those discussed above, the mass spectrometry is performed on a sample, optionally a plurality of samples, obtained from the subject or the cell/tissue culture specimen. In various embodiments, such as those discussed above, the subject is a mammal, optionally a mouse, a rat or a human. In various embodiments, such as those discussed above, the sample is or comprises urine, stool, blood or a fraction thereof, cerebrospinal fluid (CSF), saliva, muscle cell, fat cell, bone cell, hair, nail, skin cell, tumour cell or secretions, liver cell or secretions, heart cell, lung cell or secretions, pancreas cell or secretions, and/or stomach cell or secretions. In various embodiments, such as those discussed above, the sample is a frozen tissue section. In various embodiments, such as those discussed above, the sample is or comprises a cell from the cell culture or culture media from the cell culture. In various embodiments, such as those discussed above, the mass spectrometry is mass cytometry or multiplex ion beam imaging, optionally the mass cytometry is mass cytometry imaging or mass cytometry suspension.

In various embodiments, such as those discussed above, the one or more aromatic rings are 5- or 6-membered aromatic ring, optionally the one or more monocyclic 5- or 6-membered aromatic rings are independently selected from thiophene, furan, pyrrole, phenyl, or pyridine. In various embodiments, such as those discussed above, the one or more bicyclic aromatic rings are independently selected from naphthyl, indole, benzothiophene, or benzofuran. In various embodiments, such as those discussed above, the structure of the small molecule compound comprises one or more 5-membered aromatic rings, optionally the one or more 5-membered aromatic rings are each independently selected from thiophene, furan, or pyrrole. In various embodiments, such as those discussed above, the one or more 5-membered aromatic rings are thiophene. In various embodiments, such as those discussed above, the small molecule compound is teniposide and the tellurophene small molecule analogue is as shown in Formula I

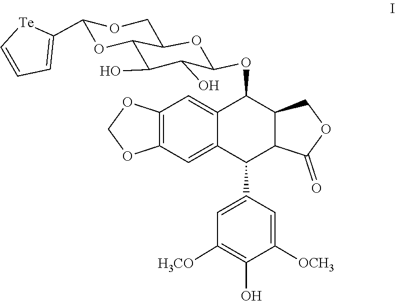

or a salt or solvate thereof.

In various embodiments, such as those discussed above, the structure of the small molecule compound comprises one or more 6-membered aromatic rings, optionally the one or more 6-membered aromatic rings are each independently selected from phenyl or pyridine, optionally the one or more 6-membered aromatic rings are phenyl. In various embodiments, such as those discussed above, the small molecule compound is carfilzomib and the tellurophene small molecule analogue is as shown in Formula II

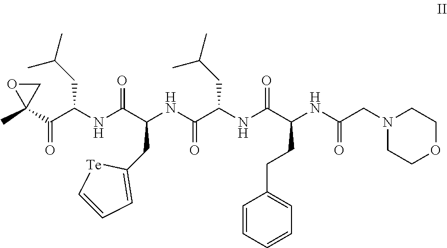

or a salt or solvate thereof.

In another aspect, the present disclosure relates to a compound of Formula I

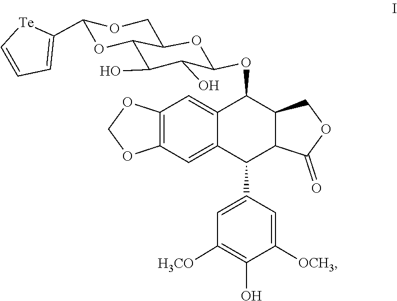

or a salt or solvate thereof.

In various embodiments, such as those discussed above, the Te atom is an isotope, optionally the Te atom is selected from ¹²⁰Te, ¹²²Te, ¹²³Te, ¹²⁴Te, ¹²⁵Te, ¹²⁶Te, ¹²⁸Te, ¹³⁰Te, and combinations thereof. In another aspect, a composition comprises a small molecule compound is carfilzomib and the tellurophene small molecule analogue is as shown in Formula II

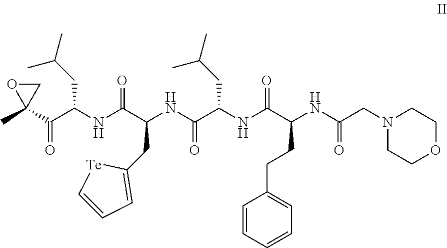

or a salt or solvate thereof and a carrier or excipient.

In another aspect, a composition comprises A compound of Formula I

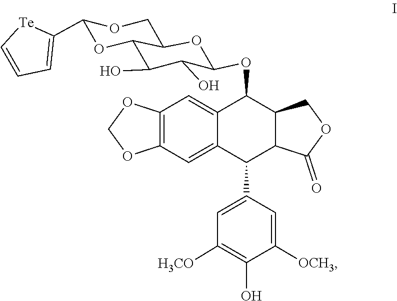

or a salt or solvate thereof and a carrier or excipient.

In various embodiments, such as those discussed above, the Te atom is selected from ¹²⁰Te, ¹²²Te, ¹²³Te, ¹²⁴Te, ¹²⁵Te, ¹²⁶Te, ¹²⁸Te, ¹³⁰Te, and combinations thereof. In yet another aspect, the present disclosure relates to a compound of Formula II

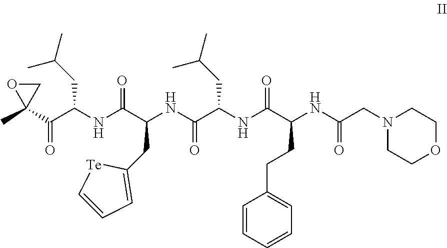

or a salt or solvate thereof.

In various embodiments, such as those discussed above, the Te atom is isotopically enriched, optionally the Te atom is selected from ¹²⁰Te, ¹²²Te, ¹²³Te, ¹²⁴Te, ¹²⁵Te, ¹²⁶Te, ¹²⁸Te, ¹³⁰Te, and combinations thereof. In another aspect, a composition comprises a compound of Formula I

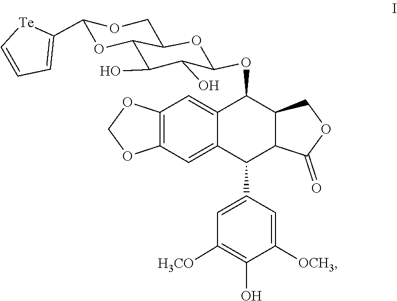

or a salt or solvate thereof and a carrier or excipient.

In another aspect, a composition comprises a compound of Formula II

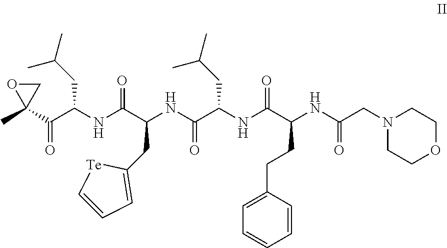

or a salt or solvate thereof and a carrier or excipient.

In another aspect, the present disclosure relates to use of a teniposide analogue in a method of detection of teniposide by mass spectrometry comprising the steps of providing a teniposide analogue, wherein the teniposide analogue is a compound of Formula I

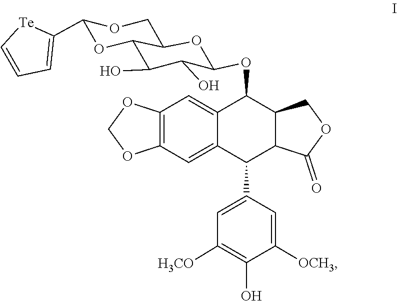

or a salt or solvate thereof, optionally wherein the Te atom is an isotope, optionally the Te atom is selected from ¹²⁰Te, ¹²²Te, ¹²³Te, ¹²⁴Te, ¹²⁵Te, ¹²⁶Te, ¹²⁸Te, ¹³⁰Te, and combinations thereof, optionally in a composition comprising at least one carrier or excipient, providing either a subject or cell/tissue culture specimen, administering the teniposide analogue to either a subject or cell/tissue culture specimen, providing a sample which is taken either from the subject or from the cell/tissue culture specimen after administration of the teniposide analogue, performing mass spectrometry on the sample to determine a level of the tellurium atom present in the sample, wherein the level of the tellurium atom corresponds to the level of teniposide analogue, and detection of the teniposide analogue is indicative of detection of teniposide in the sample.

In another aspect, the present disclosure relates to use of a carfilzomib analogue in a method of detection of carfilzomib by mass spectrometry comprising the steps of providing a carfilzomib analogue, wherein the carfilzomib analogue is a compound of formula II

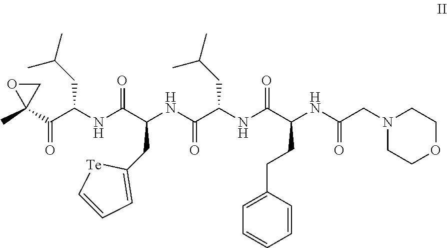

or a salt or solvate thereof, optionally wherein the Te atom is isotopically enriched, optionally the Te atom is selected from ¹²⁰Te, ¹²²Te, ¹²³Te, ¹²⁴Te, ¹²⁵Te, ¹²⁶Te, ¹²⁸Te, ¹³⁰Te, and combinations thereof, optionally in a composition comprising at least one carrier or excipient, providing either a subject or cell/tissue culture specimen, administering the carfilzomib analogue to either a subject or cell/tissue culture specimen, providing a sample which is taken either from the subject or from the cell/tissue culture specimen after administration of the carfilzomib analogue, performing mass spectrometry on the sample to determine a level of the tellurium atom present in the sample, wherein the level of the tellurium atom corresponds to the level of carfilzomib analogue, and detection of the carfilzomib small molecule analogue is indicative of detection of carfilzomib in the sample.

In various embodiments, such as those described above, the mass spectrometry is performed on a sample, optionally a plurality of samples, obtained from the subject or the cell culture. In various embodiments, such as those discussed above, the subject is a mammal, optionally a mouse, a rat or a human. In various embodiments, such as those discussed above, the sample is or comprises urine, stool, blood or a fraction thereof, cerebrospinal fluid (CSF), saliva, muscle cell, fat cell, bone cell, hair, nail, skin cell, tumour cell or secretions, liver cell or secretions, heart cell, lung cell or secretions, pancreas cell or secretions, and/or stomach cell or secretions. In various embodiments, such as those described above, the sample is a frozen tissue section. In various embodiments, such as those described above, the sample is or comprises a cell from the cell culture or culture media from the cell culture. In various embodiments, such as those described above, the mass spectrometry is mass cytometry or multiplex ion beam imaging, optionally the mass cytometry is mass cytometry imaging or mass cytometry suspension. In various embodiments, such as those described above, the tellurium atom comprises a plurality of isotopes and the mass spectrometry is multiple channel mass spectrometry, optionally multiple channel mass cytometry.

In another aspect, a kit for mass cytometry analysis comprises a tellurophene analogue of teniposide of Formula I

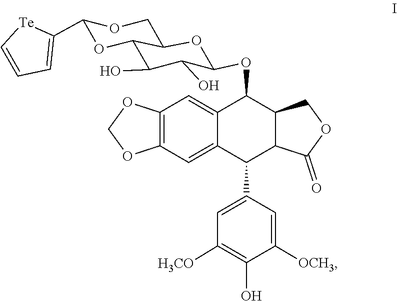

or a salt or solvate thereof, and a tellurium standard or a plurality of tellurium standards.

In another aspect, a kit for mass spectrometry analysis, optionally mass cytometry analysis comprises a tellurophene analogue of carfilzomib of Formula II

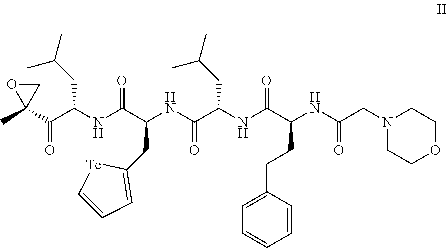

or a salt or solvate thereof, and a tellurium standard or a plurality of tellurium standards.

In various embodiments, such as those described above, the tellurium in the analogue and the tellurium in the standards are selected from ¹²⁰Te, ¹²²Te, ¹²³Te, ¹²⁴Te, ¹²⁵Te, ¹²⁶Te, ¹²⁸Te, ¹³⁰Te, and combinations thereof.

In yet another aspect, a method of determining a dosage amount of a small molecule compound to achieve a desired target engagement of the small molecule compound in a subject or a cell/tissue culture, the steps comprise providing either a subject or cell/tissue culture specimen, providing a small molecule analogue of a small molecule compound, wherein the wherein the small molecule compound has a structure comprising one or more monocyclic or bicyclic aromatic rings, and the small molecule analogue has a structure where at least one of the one or more aromatic rings of the structure of the small molecule compound is replaced with tellurophene comprising a tellurium atom, thereby forming a tellurophene small molecule analogue, administering the tellurophene small molecule analogue to the subject or cell/tissue culture specimen, providing a sample which is taken either from the subject or from the cell/tissue culture specimen after administration of the tellurophene small molecule analogue, detecting the tellurophene small molecule analogue in the sample that has been administered the tellurophene small molecule analogue, using mass spectrometry by a method as defined in any of the embodiments discussed herein, measuring the effect produced by the tellurophene small molecule analogue, and determining a dosage amount of the tellurophene analogue suitable to achieve the desired distribution of the small molecule compound, wherein the dosage amount of the tellurophene small molecule analogue indicates the dosage amount of the small molecule compound.

### DRAWINGS

The embodiments of the disclosure will now be described in greater detail with reference to the attached drawings in which:

FIG. 1 shows the effect of teniposide and Te-teniposide analogue 1 on the Top2 catalysed decatenation of kinetoplast DNA. Panel A shows a picture of the agarose gel of the Top2 decatenation assay. Panel b shows a graph of the relative Top2 inhibition based on image density of kDNA band in lane 3. 1: linear DNA; 2: decatenated kDNA; 3: control kDNA; 4. kDNA incubated with Top2; 5-8: kDNA incubated with 5, 10, 25 and 50 μM teniposide; 9-12: kDNA incubated with 5, 10, 25 and 50 μM 1.

FIG. 2 shows a graph of the relative cellular proliferation of HL-60 cells treated with teniposide or Te-teniposide analogue 1.

FIG. 3 shows a graph of the dose-dependent cytotoxicity of teniposide and analogue 1 in PANC-1 cells as determined by alamarBlue assay.

FIG. 4 shows proliferation curves of PANC-1 cells incubated with various concentrations of teniposide and 1 as determined by imaging cell confluency.

FIG. 5 shows a picture of a Westernblot of pH2AX^{Ser139}, a marker for DNA DSBs, in PANC-1 cells treated with 1 μM of drug. Teniposide and 1 exposure induced extensible DNA damage at both 24 and 48 hours relative to control.

FIG. 6 shows ¹³⁰Te histograms of cellular competitive binding assays of teniposide and compound 1 analyzed by CYTOF®. a) HL-60 cells treated with teniposide (red); Compound 1 labels HL-60 cells equally at 2 μM and 10 μM (blue and green) despite pre-saturation of drug binding sites in cells with equal amount of teniposide (orange and purple). b) Co-incubation of 2 μM teniposide with 0 (red), 2 (blue), 10 (orange) and 20 μM (green) teniposide.

FIG. 7 shows representative images of immunostained FFPE tumour tissue sections in PANC-1 xenograft mice dosed with normal (a) saline, (b) teniposide (20 mg/kg, IP) or (c) compound 1 (20 mg/kg, IP). Brown staining was done to image pH2AX^{Ser139} puncta followed by hematoxylin counterstaining.

FIG. 8 shows images of IMC™ analysis of Te-teniposide in PANC-1 xenograft. A) Teniposide dosed, B) Te-teniposide dosed, C) Image with average signal from A subtracted from B, with areas enlarged to show Te-teniposide specific signal. D) Histogram of ¹²⁵Te pixel counts from teniposide and Te-teniposide dosed mice; E) Standard curve of ¹²⁵Te counts vs Te atoms/pixel.

FIG. 9 shows a graph of percent reduction in apparent enzyme velocity when inhibited with carfilzomib, 7b, or 8b, relative to no treatment.

FIG. 10 shows a graph of median ¹²⁸Te counts per cell obtained from MC analysis of cells treated with TeCar 7b (500 μM), Carfilzomib (500 μM), control 8b, or a combination of Carfilzomib with either 7b or 8b.

FIG. 11 shows a standard curve of the in-vitro binding assay.

FIG. 12 shows a mass cytometry system and a controller.

FIG. 13 shows the mass cytometry system.

FIG. 14 shows an exemplary computer system that may serve as controller.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DESCRIPTION OF VARIOUS EMBODIMENTS

### I. Definitions

Unless otherwise indicated, the definitions and embodiments described in this and other sections are intended to be applicable to all embodiments and aspects of the present disclosure herein described for which they are suitable as would be understood by a person skilled in the art.

The term “compound(s) of the disclosure” or “compound(s) of the present disclosure” and the like as used herein refers to a tellurophene small molecule analogue as described herein. As used herein, a tellurophene small molecule analogue refers to a structural analogue of a small molecule that comprises one or more monocyclic or bicyclic aromatic rings, where the analogue comprises a tellurophene and a structure where at least one of the monocyclic or bicyclic aromatic rings is replaced by the tellurophene. As such, it can be understood that the tellurophene small molecule analogue has a structure that mimics the structure of the small molecule. For example, compounds of the present disclosure include compound of Formula I or pharmaceutically acceptable salts and/or solvates thereof, and compound of Formula II or pharmaceutically acceptable salts and/or solvates thereof.

The term “composition(s) of the disclosure” or “composition(s) of the present disclosure” and the like as used herein refers to a composition, such a pharmaceutical composition, comprising one or more compounds of the disclosure.

The term “and/or” as used herein means that the listed items are present, or used, individually or in combination. In effect, this term means that “at least one of” or “one or more” of the listed items is used or present. The term “and/or” with respect to pharmaceutically acceptable salts and/or solvates thereof means that the compounds of the disclosure exist as individual salts and hydrates, as well as a combination of, for example, a solvate of a salt of a compound of the disclosure.

As used in the present disclosure, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

As used in this disclosure and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

In embodiments of the present disclosure, the compounds described herein may have at least one asymmetric center. Where compounds possess more than one asymmetric center, they may exist as diastereomers. It is to be understood that all such isomers and mixtures thereof in any proportion are encompassed within the scope of the present disclosure. It is to be further understood that while the stereochemistry of the compounds may be as shown in any given compound listed herein, such compounds may also contain certain amounts (for example, less than 20%, suitably less than 10%, more suitably less than 5%) of compounds of the present disclosure having an alternate stereochemistry. It is intended that any optical isomers, as separated, pure or partially purified optical isomers or racemic mixtures thereof are included within the scope of the present disclosure.

The compounds of the present disclosure may also exist in different tautomeric forms and it is intended that any tautomeric forms which the compounds form, as well as mixtures thereof, are included within the scope of the present disclosure.

The compounds of the present disclosure may further exist in varying polymorphic forms and it is contemplated that any polymorphs, or mixtures thereof, which form are included within the scope of the present disclosure.

The present description refers to a number of chemical terms and abbreviations used by those skilled in the art. Nevertheless, definitions of selected terms are provided for clarity and consistency.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms.

The term “available”, as in “available hydrogen atoms” or “available atoms” refers to atoms that would be known to a person skilled in the art to be capable of replacement by a substituent.

The term “amine” or “amino,” as used herein, whether it is used alone or as part of another group, refers to groups of the general formula NR′R″, wherein R′ and R″ are each independently selected from hydrogen or C₁₋₆alkyl.

The term “atm” as used herein refers to atmosphere.

The term “MS” as used herein refers to mass spectrometry.

The term “aq.” as used herein refers to aqueous.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “small molecule” as used herein refers to a low molecular weight organic compound. For example, the small molecule can have a molecular weight of less than about 1500 g/mol, less than about 1300 g/mol, or less than about 1000 g/mol.

The term “subject” as used herein includes all members of the animal kingdom including mammals, and suitably refers to rats, mice and humans. Thus, the methods and uses of the present disclosure are applicable to both human therapy and veterinary applications.

The term “pharmaceutically acceptable” means compatible with the treatment of subjects.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “solvate” as used herein means a compound, or a salt of a compound, wherein molecules of a suitable solvent are incorporated in the crystal lattice

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the disclosure to prevent recurrence. Treatment methods comprise administering to a subject a therapeutically effective amount of one or more of the compounds of the disclosure and optionally consist of a single administration, or alternatively comprise a series of administrations.

As used herein, the term “effective amount” or “therapeutically effective amount” means an amount of one or more compounds of the disclosure that is effective, at dosages and for periods of time necessary to achieve the desired result.

The term “administered” as used herein means administration of a therapeutically effective amount of one or more compounds or compositions of the disclosure to a cell, tissue, organ or subject.

The term “neoplastic disorder” as used herein refers to a disease, disorder or condition characterized by cells that have the capacity for autonomous growth or replication, e.g., an abnormal state or condition characterized by proliferative cell growth. The term “neoplasm” as used herein refers to a mass of tissue resulting from the abnormal growth and/or division of cells in a subject having a neoplastic disorder.

The term “cancer” as used herein refers to cellular-proliferative disease states.

### II. Compounds and Compositions of the Disclosure

In one aspect, the present disclosure includes a tellurophene small molecule analogue of a small molecule, wherein the tellurophene small molecule analogue comprises a tellurophene, the tellurophene comprising a tellurium atom, wherein the small molecule has a structure comprising one or more monocyclic or bicyclic aromatic rings, and the analogue has a structure where at least one of the one or more aromatic rings of the structure of the small molecule is replaced with the tellurophene.

In some embodiments, the small molecule is any organic compound that produces a physiological effect in a subject that has been administered the organic compound. For example, the small molecule is a drug. In some embodiments, the small molecule is not phenylalanine, and the tellurophene small molecule analogue is not TePhe.

In some embodiments, the one or more monocyclic aromatic rings are 5- or 6-membered aromatic ring, optionally the one or more monocyclic 5- or 6-membered aromatic rings are independently selected from thiophene, furan, pyrrole, phenyl, or pyridine.

In some embodiments, the one or more bicyclic aromatic rings are independently selected from naphthyl, indole, benzothiophene, or benzofuran.

In some embodiments, the structure of the small molecule comprises one or more monocyclic 5- or 6-membered aromatic rings and at least one of the monocyclic 5- or 6-membered aromatic rings is replaced with the tellurophene.

In some embodiments, the structure of the small molecule comprises one or more comprise bicyclic aromatic rings, and the at least one of the bicyclic aromatic rings is replaced with a benzo[b]tellurophene or a 4H-telluropheno[3,2-b]pyrrole.

In some embodiments, the structure of the small molecule comprises one or more 5-membered aromatic rings, optionally the one or more 5-membered aromatic rings are each independently selected from thiophene, furan, or pyrrole.

In some embodiments, the one or more 5-membered aromatic rings are thiophene.

In some embodiments, the structure of the small molecule comprises one or more 6-membered aromatic rings, optionally the one or more 6-membered aromatic rings are each independently selected from phenyl or pyridine, optionally the one or more 6-membered aromatic rings are phenyl.

It is contemplated that tellurophene analogues of small molecules such as therapeutics comprising one or more aromatic rings can be designed and prepared by replacing at least one of the one or more aromatic rings. As such, it is contemplated that the structure of the analogue mimics the structure of the small molecule such that the analogue exhibits substantively similar biological activity as the small molecule. Thus, detection of the analogue is indicative of the detection of the small molecule. Some non-limiting examples are provided in Scheme A below.

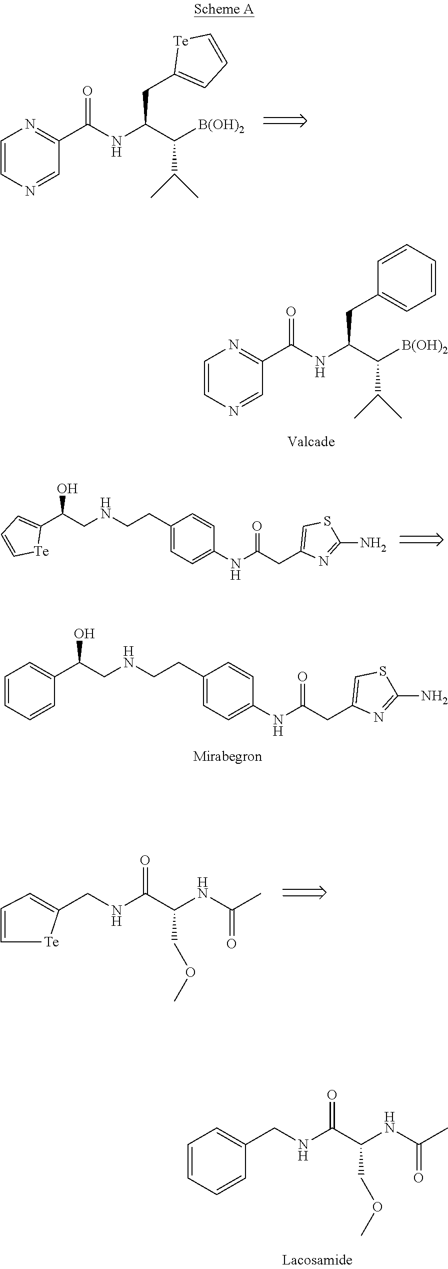

In some embodiments, the small molecule is teniposide and the tellurophene small molecule analogue is as shown in Formula I

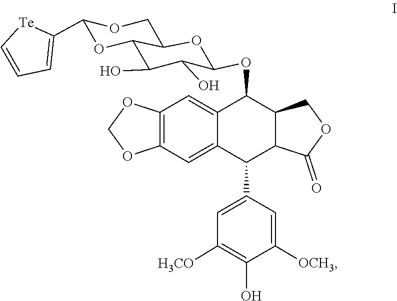

or a salt or solvate thereof.

In some embodiments, the small molecule is carfilzomib and the analogue is as shown in Formula II

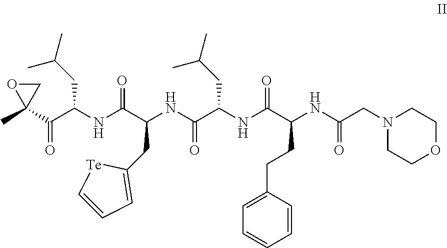

or a salt or solvate thereof.

In another aspect, the present disclosure includes a compound of Formula I

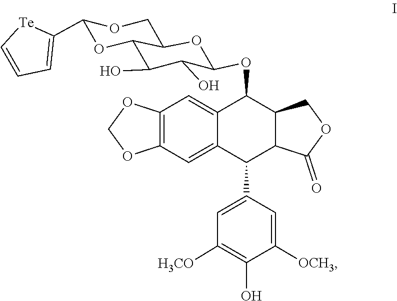

or a salt or solvate thereof.

In another aspect, the present disclosure includes a compound of Formula II

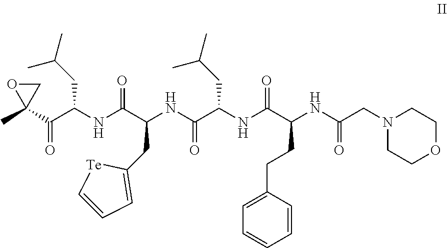

or a salt or solvate thereof.

In another aspect, the present disclosure includes a composition comprising the compound of the present disclosure and a carrier or excipient.

In an embodiment, the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the disclosure for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of compounds of the disclosure include, for example, those made with solvents that are pharmaceutically acceptable. Examples of such solvents include water (resulting solvate is called a hydrate) and ethanol and the like. Suitable solvents are physiologically tolerable at the dosage administered.

The compounds of the present disclosure are suitably formulated in a conventional manner into compositions using one or more carriers. Accordingly, the present disclosure also includes a composition comprising one or more compounds of the disclosure and a carrier. The compounds of the disclosure are suitably formulated into pharmaceutical compositions for administration to subjects in a biologically compatible form suitable for administration in vivo. Accordingly, the present disclosure further includes a pharmaceutical composition comprising one or more compounds of the disclosure and a pharmaceutically acceptable carrier. In embodiments of the disclosure the pharmaceutical compositions are used in the treatment of any of the diseases, disorders or conditions described herein.

The compounds of the disclosure are administered to a subject in a variety of forms depending on the selected route of administration, as will be understood by those skilled in the art. For example, a compound of the disclosure is administered by oral, inhalation, parenteral, buccal, sublingual, nasal, rectal, vaginal, patch, pump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a compound of the disclosure is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the disclosure is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the disclosure and use the lyophilizates obtained, for example, for the preparation of products for injection.

In some embodiments, a compound of the disclosure is administered parenterally. For example, solutions of a compound of the disclosure are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the disclosure are usually prepared, and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, a compound of the disclosure is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, the compounds of the disclosure are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of the disclosure are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of the disclosure and a suitable powder base such as lactose or starch. The aerosol dosage forms can also take the form of a pump-atomizer.

Compositions suitable for buccal or sublingual administration include tablets, lozenges, and pastilles, wherein a compound of the disclosure is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the disclosure are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

In some embodiments a compound of the disclosure is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of the disclosure is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

A compound of the disclosure including pharmaceutically acceptable salts and/or solvates thereof is suitably used on their own but will generally be administered in the form of a pharmaceutical composition in which the one or more compounds of the disclosure (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

### III. Methods and Uses of the Disclosure

In one aspect, the present disclosure includes a method of detecting of a small molecule within a sample, the method comprising
providing a sample obtained from a subject or from a cell/tissue culture that has been administered a tellurophene small molecule analogue comprising a tellurophene, the tellurophene comprising a tellurium atom; and
performing mass spectrometry on the sample to determine a level of the tellurium atom;
   wherein the small molecule has a structure comprising one or more monocyclic or bicyclic aromatic rings, and the analogue has a structure where at least one of the one or more aromatic rings of the structure of the small molecule is replaced with the tellurophene, and wherein the level of the tellurium atom corresponds to the level of the analogue and detection of the analogue is indicative of detection of the small molecule in the sample.

In some embodiments, the method further comprises quantifying the amount of the tellurium atom in the sample, wherein the tellurium atom level is indicative of the small molecule quantity in the sample.

In some embodiments, the method further comprises quantifying one or more other analytes within the sample, the method further comprising labelling the sample with one or more mass tagged analyte binders prior to performing mass spectrometry, and determining a level of the one or more analyte binders.

In some embodiments, the one or more mass tagged analyte binders are selected from metal-labelled antibodies, polymer-labelled antibodies, metal-labelled oligonucleotides, polymer-labelled oligonucleotides, intercalators such as 5-iodo-2′-deoxyuridine (IdU), and metal-containing intercalators (e.g. Rh-containing intercalator, and Ir-containing intercalator), metal-containing viability indicator such as cisplatin, barcoding reagents such as Cd-labelled CD45 and Pt-labelled CD45. In some embodiments, the polymer-labelled antibodies comprise metal. For example, the polymer can be metal-bound.

In some embodiments, the subject is a mammal, optionally a mouse, a rat or a human.

In some embodiments, the mass spectrometry is performed at a plurality of discrete locations and the level of the tellurium atom is determined at each of the plurality of discrete locations to provide a distribution of the analogue within the sample, and wherein the distribution of the analogue within the sample is indicative of a distribution of the small molecule within the sample.

In some embodiments, the sample is a single cell and the distribution of the analogue within the single cell is indicative of a distribution of the small molecule at a subcellular level.

In some embodiments, a plurality of samples is provided and the samples comprise different tissues, cells or secretions of the subject, and wherein the detection of the analogue within the plurality of samples provides a distribution of the analogue within the subject which is indicative of a distribution of the small molecule within the subject, such as tissue or organ distribution.

In some embodiments, the sample is or comprises urine, stool, blood or a fraction thereof, cerebrospinal fluid (CSF), saliva, muscle cell, fat cell, bone cell, hair, nail, skin cell, tumour cell or secretions, liver cell or secretions, heart cell, lung cell or secretions, pancreas cell or secretions, and/or stomach cell or secretions.

In some embodiments, the sample is a frozen tissue section.

In some embodiments, the sample is or comprises a cell from the cell culture or culture media from the cell culture.

In some embodiments, the sample is a single cell and the distribution of the analogue within the single cell is indicative of a distribution of the small molecule at a subcellular level.

In some embodiments, the analogue interacts irreversibly, optionally covalently, with a target in the sample.

In some embodiments, the subject or the cell/tissue culture had been administered the tellurophene small molecule analogue in combination with the small molecule, wherein the method further comprises detecting directly a level of the small molecule, and wherein a comparison of the level of the small molecule and the level of the analogue is indicative of a putative competitive binding of the analogue.

In another aspect, the present disclosure includes a use of a tellurophene analogue in the detection of a small molecule by mass spectrometry in a subject that has been administered the analogue or a cell/tissue culture that has been administered the analogue, wherein the small molecule has a structure comprising one or more monocyclic or bicyclic aromatic rings, and the analogue has a structure where at least one of the one or more monocyclic or bicyclic aromatic rings of the structure of the small molecule is replaced with a tellurophene.

In another aspect, the present disclosure includes a use of the compound of Formula I, or the composition comprising the compound of Formula I of the present disclosure in the detection of teniposide by mass spectrometry in a subject that has been administered the compound of Formula I or salt or solvate thereof or a cell/tissue culture that has been administered the compound of Formula I or salt or solvate thereof.

In another aspect, the present disclosure includes a use of the compound of Formula II, or the composition comprising the compound of Formula II of the present disclosure in the detection of carfilzomib by mass spectrometry in a subject that has been administered the compound of Formula II or salt or solvate thereof or a cell/tissue culture that has been administered the compound of Formula II or salt or solvate thereof.

In another aspect, the present disclosure includes a kit for mass cytometry analysis comprising a tellurophene analogue of teniposide of Formula I

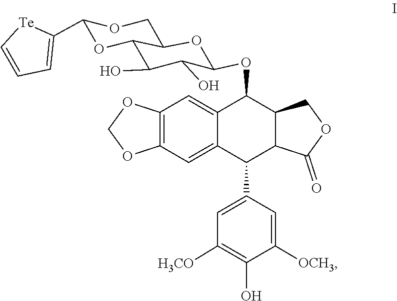

or a salt or solvate thereof, and
a tellurium standard or a plurality of tellurium standards.

In another aspect, the present disclosure includes a kit for mass spectrometry analysis, optionally mass cytometry analysis comprising a tellurophene analogue of carfilzomib of Formula II

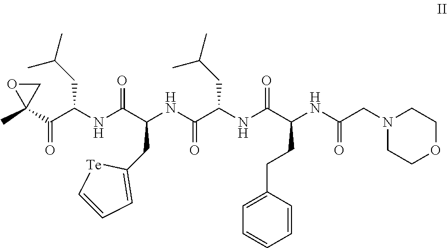

or a salt or solvate thereof, and
a tellurium standard or a plurality of tellurium standards.

In another aspect, the present disclosure includes a method of determining a dosage amount of a small molecule to achieve a desired target engagement of the small molecule in a subject or a cell/tissue culture, wherein the small molecule engages a target in the subject or the cell/tissue culture and produces a measurable effect at the target, the method comprising
detecting the small molecule in a subject by a method of the present disclosure;
measuring the effect produced by the small molecule; and
determining a dosage amount of the tellurophene analogue suitable to achieve the desired distribution of the small molecule, wherein the dosage amount of the tellurophene analogue indicates the dosage amount of the small molecule.

Mass cytometry, in addition to enabling single cell analysis can include mass cytometry imaging methods for example as described in (Giesen et al 2014, incorporated herein by reference). In such methods, a tissue or cell population is labelled in vitro with mass-tagged entities such as analyte binders and/or compounds comprising suitable atoms for mass cytometry, the tissue or cell population is subjected to laser ablation coupled to mass cytometry and the tellurium signal processed to provide an image showing single cell segmentation. Different tissue preparations can be used including for example formalin fixed and fresh tissue.

In some embodiments, the mass spectrometry is mass cytometry or multiplex ion beam imaging, optionally the mass cytometry is mass cytometry imaging or mass cytometry suspension.

In some embodiments, the tellurium atom is selected from ¹²⁰Te, ¹²²Te, ¹²³Te, ¹²⁴Te, ¹²⁵Te, ¹²⁶Te, ¹²⁸Te, ¹³⁰Te, and combinations thereof.

In some embodiments, the tellurium atom comprises a plurality of tellurium isotopes and the mass cytometry is multichannel mass spectrometry, optionally multichannel mass cytometry.

It can be appreciated that the methods of the present disclosure and the tellurophene analogues of the present disclosure can be used to track drug distribution in organs or tissues in general. For example, the methods can be used to assess small molecule distribution when the small molecule has been administered through different routes of administration (e.g. inhalation, injection, etc.).

It is contemplated that the tellurophene analogues of the present disclosure and the methods of the present disclosure can be used in combination, contemporaneously or sequentially with other analytical methods and reagents. For example, the tellurophene analogues of the present disclosure and the methods of the present disclosure can be used in combination, contemporaneously or sequentially with labelled-antibodies (e.g. metal-labelled antibodies and polymer-labelled antibodies) such as MAXPAR® reagents, metal-labelled oligonucleotides, metal-containing intercalators and viability indicators (e.g. metal viability indicators) such as CELL-ID™ reagents, barcoding reagents such as Cell-ID™ 20-Plex Pd Barcoding Kit; Cd-CD45; Pt-CD45, and/or calibration beads such as EQ™ Four Element Calibration Beads and EQ™ Six Element Calibration Beads.

It is also contemplated that tellurophene analogues of the present disclosure and the methods of the present disclosure can be used in different applications including diagnostics, preclinical studies including animal studies, pharmacokinetics (e.g. pulse-chase studies) and pharmacodynamics studies.

It is contemplated that depending on the nature of the small molecule and the analogue thereof, the methods of the present disclosure and the analogue of the present disclosure can be used to study various conditions including neoplastic disorders such as leukaemia, neuroblastoma, and Non-Hodgkin's lymphoma.

### IV. Methods of Preparing the Compounds of the Disclosure

Compounds of the present disclosure can be prepared by various synthetic processes. The choice of particular structural features and/or substituents may influence the selection of one process over another. The selection of a particular process to prepare a given compound of the present disclosure is within the purview of the person of skill in the art. Some starting materials for preparing compounds of the present disclosure are available from commercial chemical sources. Other starting materials, for example as described below, are readily prepared from available precursors using straightforward transformations that are well known in the art. In the Schemes below showing the preparation of compounds of the disclosure, all variables are as defined herein, unless otherwise stated.

The formation of a desired compound salt is achieved using standard techniques. For example, the neutral compound is treated with an acid or base in a suitable solvent and the formed salt is isolated by filtration, extraction or any other suitable method.

The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”. The formation of solvates of the compounds of the disclosure will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

Accordingly, in some embodiments, the compounds of the present disclosure can be prepared as described below. For example, the compound of Formula I can be prepared as shown in Scheme 1. For example, the compound of Formula II can be prepared as shown Scheme 2. It can be appreciated that tellurophene moiety can be incorporated into small molecules to replace one or more aromatic rings by other suitable synthetic approaches. Some suitable examples are provided in the schemes below.

### Methods of Appending Tellurophene

In some embodiments, tellurophene can be incorporated via an amine group. For example, tellurophenyl amines can be obtained from tellurophene aldehyde through reductive amination as shown in Scheme B.

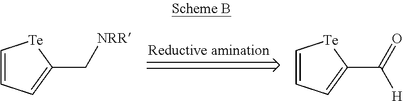

In some embodiments, tellurophene can be incorporated into a small molecule via a carboxyl group. For example, tellurophene carboxylic acid can be obtained from tellurophene aldehyde through oxidation. The resulting tellurophene carboxylic acid can be coupled to a small molecule via an amide or an ester linkage. (Scheme C)

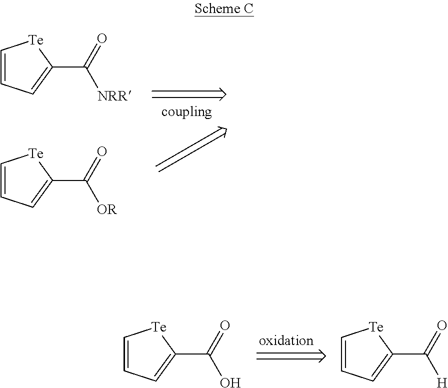

In some embodiments, tellurophene can be transformed into tellurophene halide by halogenation, which can be incorporated into a small molecule through metal-catalysed cross-coupling reactions. (Scheme D)

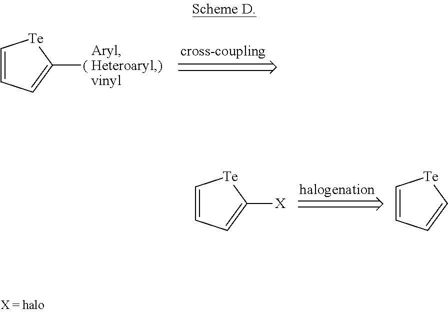

It is also contemplated that other tellurophene-containing heterocycles (e.g. bicyclic heterocycles) can be can be used to replace aromatic rings in small molecules. For example, an analogue of a small molecule comprising a bicyclic aromatic ring can be designed and prepared by replacing the bicyclic aromatic ring with a bicyclic tellurophene containing heterocycle. In some embodiments, a benzofuran or indole can be replaced with a benzo[b]tellurophene. Benzo[b]tellurophenes can be prepared by cyclization of alkynyl arylhalide in the presence of tellurium. (Scheme E)

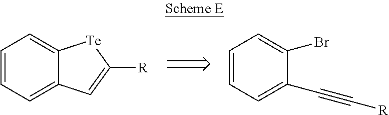

In some embodiments, a thieno[3,2-b]pyrrole and/or a pyrrolo[3,2-b]pyrrole can be replaced with a telluropheno[3,2-b]pyrrole. For example, telluropheno[3,2-b]pyrrole can be obtained from cyclization of alkynyl pyrrolohalide. (Scheme F)

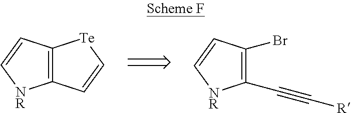

### EXAMPLES

The following non-limiting examples are illustrative of the present disclosure.

### Example 1 Tellurophene Teniposide Analogue: Synthesis and Evaluation

A tellurium-substituted analogue (compound of Formula I) of the anti-cancer xenobiotic teniposide has been developed. Teniposide is a semi-synthetic podophyllototoxin which inhibits topoisomerase II (Top2) enzymatic activity by binding to Top2-DNA complexes. Top2 modifies DNA topology by introducing transient double-stranded breaks via a covalent Top2-DNA complex. Upon binding, teniposide stabilizes the DNA-Top2 complex and prevents relegation of DNA, thus leading to a buildup of double stranded breaks (DSBs). The cytotoxic effects of teniposide are associated with apoptosis resulting from excessive DSBs. Teniposide is a clinically approved chemotherapeutic used primarily for the treatment of acute lymphocytic leukemia (ALL), Hodgkin's lymphoma and neuroblastoma. The high copy number of Top2 (1×10⁶ in transformed cell lines) suggests tellurium labelled Teniposide analogues bound to Top2 should be detectable by MC.

Teniposide bears a thiophene ring that was hypothesized to be amenable to synthetic substitution. Through synthesis of a teniposide derivative where the thienyl group was substituted with a tellurophene ring an MC visible analog was readily accessed. The Te-Teniposide analogue was demonstrated to be indistinguishable from Teniposide in cell-based assays. Further, it was shown that MC can be used to follow localization of the compound in cells. In vivo Te-teniposide behaved similarly to Teniposide and was detected directly in tissue sections.

### Results and Discussion

### Synthesis of Te-Teniposide

Using the semisynthetic strategy, a Te-teniposide analog (1) was initiated from 4′-demethylepipodophyllotoxin α-D-glucopyranoside, which could be conveniently obtained by mild acid hydrolysis of Etoposide. Formation of the desired telurophenylidene acetal was initially envisioned to proceed directly from the corresponding diethylacetal 3, as is often accomplished for benzylidene acetal formation with the corresponding dimethyl acetals. The diethyl acetal 3 was generated from propargylaldehyde diethyl acetal and (bromoethynyl)triisopropylsilane under Cadoit-Chodkiewicz conditions. The resulting diyne 2 was cyclized into the tellurophene 3 by treatment with sodium hydrogen telluride generated in situ from tellurium metal and sodium borohydride.

Using tellurophene 3, a series of model condensation reactions with methyl-α-D-glucopyranoside were investigated. The common conditions used for installation of benzylidene acetals (TsOH, CH₃CN or DMF) failed and resulted in recovery of the tellurophene aldehyde 4. The literature methods for generation of teniposide and similar acetals suggested conditions using neat aldehyde and freshly fused ZnCl₂. Tellurophene diethyl acetal 3 could be cleanly converted to the aldehyde 4 with mild acid hydrolysis. Condensing 4′-demethylepipodophyllotoxin with neat tellurophene aldehyde 4 led to the desired teniposide analogue 1 in modest yield (Scheme 1)

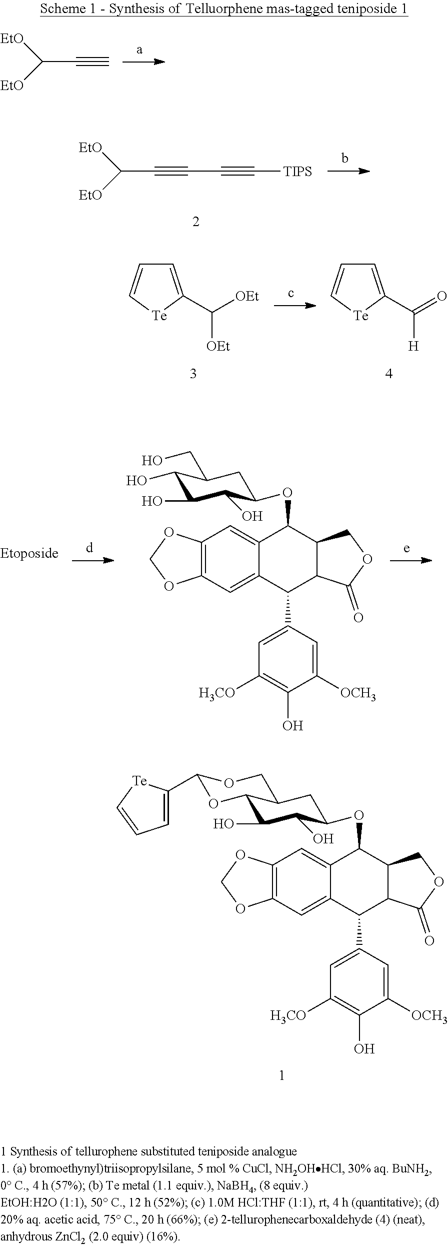

### In-Vitro Analysis of Te-Teniposide

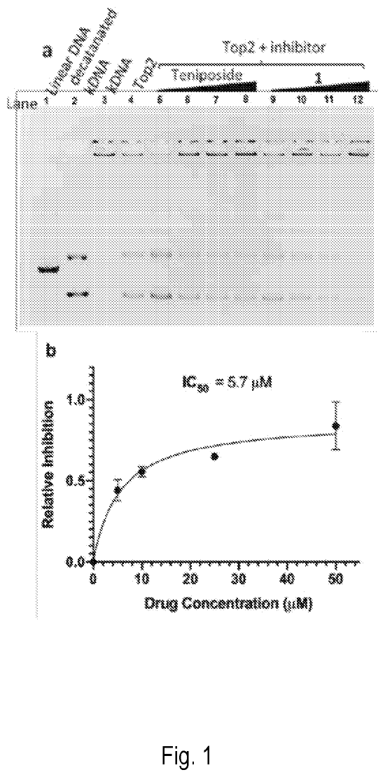

The relative Top2 inhibitory activity of 1 in comparison to teniposide was evaluated in decatenation assays using kinetoplast DNA (kDNA). Decatenation of the DNA, catalyzed by Top2, can be readily resolved using agarose gel electrophoresis. Both teniposide and 1 demonstrated dose-dependent inhibition of the decatenation reaction (FIG. 1). The calculated IC₅₀ value for 1 (5.6 μM) is similar to that of teniposide calculated from fluorescence anisotropy and DNA relaxation gel assays in literature (1.3-9.8 μM).

### Cytotoxicity of Te-Teniposide

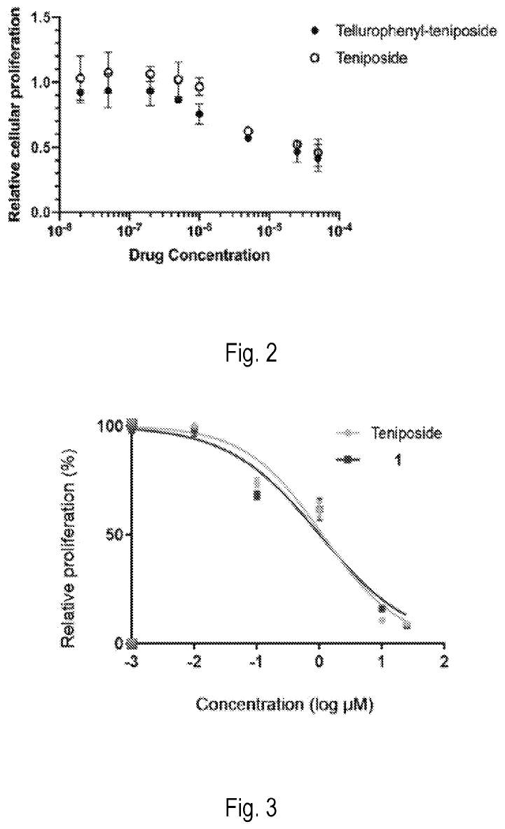

The HL-60 promyelocytic leukemia cell line has been used as a model system to understand spatial and temporal distribution of Top2 expression and its related drug resistance mechanisms in cellulo. The toxicity of 1 was compared to teniposide in HL-60 cells after incubation for 24 hours using a WST-1 viability assay. The dose response curves for teniposide and 1 were observed to be identical within experimental variability (FIG. 2.); consistent with the literature both Top2 poisons inhibited proliferation of HL-60 cells up to 60% at the maximum concentration of 50 μM.

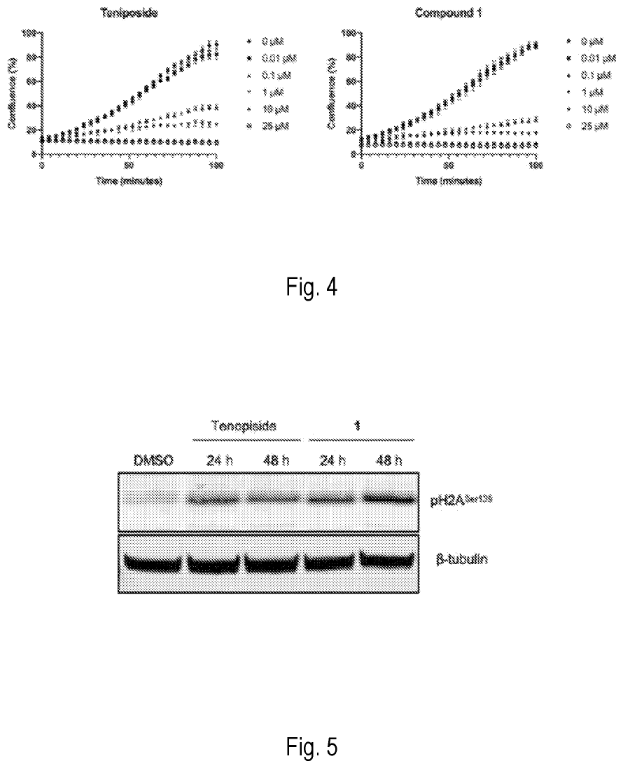

Similarly, PANC1 cells were characterized for later in vivo experiments. Using an alamarBlue viability assay and a proliferative assay, PANC-1 cells were incubated with 1 or teniposide. Both drug forms exhibited concentration dependent cytotoxicity in PANC-1 cells with indistinguishable dose response and almost complete loss of cell viability at the maximum dose of 25 μM (FIG. 3). In proliferative assays time course analysis over a 100-hour incubation revealed concentrations above 0.1 μM of teniposide or 1 resulted in substantial reduction in PANC-1 cell proliferation for both compounds (FIG. 4 panels A and B). Taken together, these data suggest 1 effectively mimics the antiproliferative activity of teniposide in PANC-1 cells.

### Te-Teniposide Induces DNA Damage

Next, the ability of 1 to induce DSBs—the dominant type of DNA damage resulting from Top2 inhibition was investigated. In response to DSBs, a cascade of DNA repair mechanisms are triggered, including-phosphorylation of the histone variant H2AX at Ser139. Thus, the level of pH2AX^{Ser139} serves as a reliable marker for Top2-linked DSBs. Western blotting was used to measure pH2AX^{Ser139} in PANC-1 cells treated with 1 μM of either 1 or teniposide for 24 and 48 hours. Increased levels of pH2AX^{Ser139} were clearly observed for both inhibitors relative to DMSO-treated control, indicating 1 exhibits parity to teniposide in its ability to induce DSBs (FIG. 5).

### Cellular Uptake Te-Teniposide

To determine if cellular labelling with 1 was dependent on the presence of Top2-DNA binding sites a competitive binding MC experiment was carried out using Teniposide. HL-60 cells were treated with teniposide (10 μM) for two hours to saturate intracellular Top2-DNA ternary complexes with an unlabelled ligand, next 1 (2 or 10 μM) was introduced into the cell media and the suspension was gently mixed. After two hours, cells were harvested and washed three times with PBS. Cells were then fixed and permeabilized, followed by DNA staining with Ir-intercalator. Cell pellets were analyzed by MC for ¹³⁰Te content (FIG. 6).

While cell labelling with 1 was readily detected at both concentrations (2 and 10 μM), the expected reduction of ¹³⁰Te signal in cells pretreated with unlabeled teniposide was not observed (FIG. 6A). Additionally, co-incubation experiments were conducted where HL-60 cells were treated with a cocktail of 1 (2 μM) and either 2, 10, or 20 μM teniposide (FIG. 6B). Population histograms of all three treatment groups were identical suggesting no competition for Top2 could be observed under these conditions. Without wishing to be bound by theory, it is hypothesized that the lack of competitive labelling observed in these experiments may be due to a combination of inhibition of Top2 and a considerable degree of non-specific binding with other cellular components. Several reports have documented the non-specific accumulation of etoposide and teniposide via oxidation catalyzed by cytochrome P450-dependent monooxygenases, peroxidases and tyrosinase enzymes. These redox transformations generate catechol and quinone metabolites which significantly impair DNA binding ability of Top2. It is also plausible these reactive species modify other intracellular proteins, thus contributing to drug accumulation inside cells, but in a promiscuous and irreversible manner. These results exemplify the utility of MC-visible probes in following the specificity of pharmaceutically active compounds. In this case, the non-specific binding may dominate the target binding of teniposide, and likely its analogue Te-teniposide 1.

### In-Vivo Evaluation of Tellurium-Labelled Teniposide

Despite being unable to demonstrate selective reversible binding in cell suspension by MC, the ability to readily detect compound 1 and the promising activity of compound 1 in cell culture was demonstrated. Visualizing non-specific accumulation and lack of target specificity in tissue sections would improve the understanding of a therapeutic agent's distribution as has been observed with cisplatin.⁸

IMC™ can be used to image tissue localization and quantify the concentration of 1 in tissue samples. For in vivo studies, PANC-1 tumour xenograft-bearing mouse models were selected. The tumour microenvironment and general tissue morphology in sections derived from this human pancreatic cancer model can be characterised.

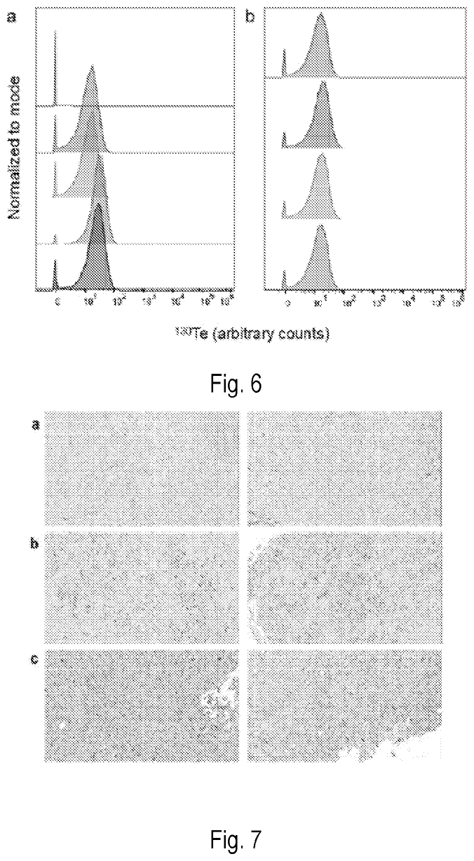

PANC-1 xenograft-bearing mice were injected with either teniposide or the Te-teniposide analogue 1 IP. A dose of 20 mg/kg was selected based on literature reports detailing teniposide drug delivery strategies. After 2 hours, mice were sacrificed and tissues of interest were harvested, formalin fixed, and paraffin embedded. Tissue sections (5 um) were cut and mounted onto microscopy slides. The expression of pH2AX^{Ser139} levels were first assessed by immunohistochemistry. Serial sections derived from PANC-1 xenograft tumours were stained with primary antibody and subsequently HRP-conjugated secondary antibody for standard brown staining. DSBs are indicated by large, discrete pH2AX^{Ser139} foci. Representative images of immunostained slides show increased pH2AX^{Ser139} levels in treated mice relative to saline injected control with clear, punctate staining (FIG. 7). These results indicate 1 was distributed across the tumour tissue and maintained its ability to induce DSBs in vivo in a similar fashion to teniposide.

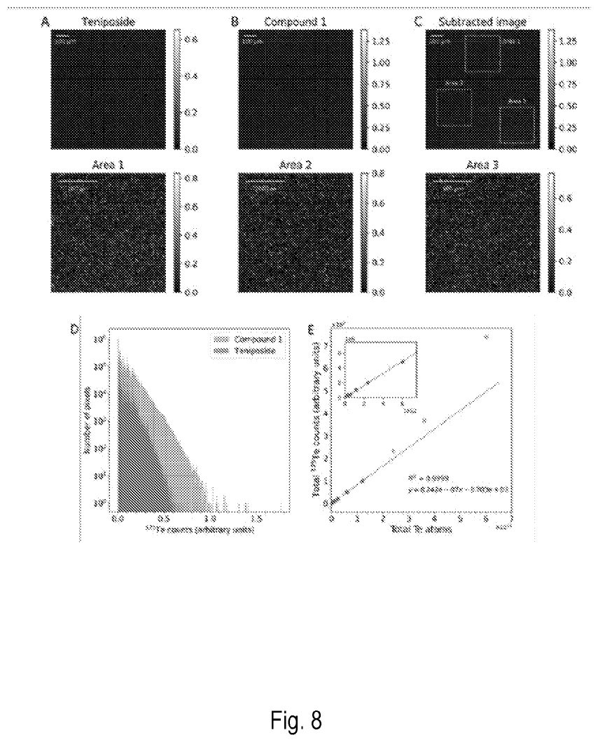

For IMC™ analysis, the initial objective was to visualize tellurium signal distribution in tumour sections and subsequently measure relative tellurium content in individual cells. Tumour sections were dewaxed, rehydrated and washed. Slides then were stained with Ir-intercalator to visualize cell nuclei, and then imaged by IMC™. The ¹²⁵Te isotope was visualised. Unfortunately, no Te signal above background could be detected in tumour sections. Efforts to combine intensities from multiple tellurium channels to improve the signal-to-noise ratio were also unsuccessful. Without wishing to be bound by theory, it was hypothesized that the processing of the FFPE tissues contributes to loss of tellurium signal as the teniposide interaction with the cells is largely non-covalent, thus the multiple wash steps required for dewaxing (xylene and ethanol washes) might have removed bound 1. To reduce the number of tissue processing steps, snap frozen samples prepared from the same tumours were sectioned and analyzed by IMC™. Using this approach, ¹²⁵Te signals were observed in the tissue samples which had been exposed to Te-teniposide 2 h before sacrifice (FIG. 8). In samples collected at later timepoints after dosing, the Te signal was not observed in line with the half-life of teniposide of 1-5 hours in mice depending on the route of administration and dosing regimen.²⁴

To determine the corresponding concentration of Te present in the sample a set of standards containing increasing concentrations of Te were analyzed by IMC™. Based on the total counts present in each standard Te spot a count/Te atom constant was calculated (FIG. 8E). The counts observed in the frozen samples suggest that the average Te-teniposide concentration in the tissue was in the low tens of micromolar at the time of analysis. The low signal observed made it impossible to calculate a precise value. The observed signal being marginally over background is consistent with the expected sensitivity of the IMC™ instrument to tellurium.

### Conclusion

The ability to directly quantify the cellular distribution of pharmacologically active small molecules in tissues using IMC™ would provide cell specific information about target engagement and biodistribution. Using the antineoplastic agent, teniposide, as shown, it is possible to generate an analogue which has indistinguishable bioactivity to the native compound through the substitution of a thienyl for a tellurenyl functional group. Using the Te-teniposide analogue, high levels of non-specific binding of the compound to cells were demonstrated by MC, suggesting the known target of teniposide, Top2, represents only a small fraction of the binding sites for the compound. This is consistent with the high level of protein binding of teniposide. In vivo, the activity of Te-teniposide generated the expected DSB in a PANC-1 xenograft, and the Te signal could be observed within the tissue.

### Experimental Section

### Chemical Synthesis

General materials and methods: Etoposide (CarboSynth), propargylaldehyde diethyl acetal (TCI America) and glacial acetic acid (Caledon Laboratories Ltd) were purchased from indicated vendors. All other reagents were purchased from Sigma Aldrich. All reactions were conducted under a dry argon atmosphere using oven-dried glassware. Absolute ethanol was obtained from Green Field Specialty Alcohols Inc. whereas all other anhydrous solvents were purchased from Sigma Aldrich and dried over 4 Å molecular sieves prior to use. Purifications by flash column chromatography used silica gel (SiliCycle Silica-P Flash Silica Gel, 40-60 um, 60 Å pore size) and gradient solvent mixtures as described. Compounds were purified using a BIOTAGE ISOLERAT system. Final compound 1 purity was confirmed using RP-HPLC coupled with low-resolution mass spectra (ESI) collected on an AGILENT™ Technologies 1200 series HPLC paired to a 6130 Mass Spectrometer. Final compound 1 was resolved on Phenomenex's KINETEX™ 2.6 um C18 50×4.6 mm column at room temperature with a flow of 1 mL/min. The gradient consisted of eluents A (0.1% formic acid in double distilled water) and B (0.1% formic acid in HPLC-grade acetonitrile). A linear gradient starting from 5% of B to 95% over 7 min at a flow rate of 1.0 mL/min. 1H and ¹³C NMR spectra were recorded on Bruker 400 MHz and Agilent 500 MHz spectrometers in CDCl₃, CD₃OD and C₂D₆SO. Chemical shifts (δ) are reported in parts per million (ppm) after calibration to the internal reference solvent peak CDCl₃: 7.26 ppm and C₂D₆SO: 2.49 ppm, CD₃OD: 3.31 ppm). Coupling constants (J) are reported in Hz. High resolution mass spectra were obtained on a VG 70-250S (double focusing) mass spectrometer at 70 eV or on an ABI/SCIEX QSTAR™ mass spectrometer with ESI source and accurate mass capabilities.

(Bromoethynyl)triisopropylsilane: To a solution of (triisopropylsilyl) acetylene (2.0 mL, 8.9 mmol, 1.0 equiv.) in acetone (0.15 M), N-Bromosuccinimide (1.75 g, 9.8 mmol, 1.1 equiv.) and silver nitrate (0.15 g, 0.89 mmol, 0.1 equiv.) were successively added. The reaction mixture was stirred at room temperature for 5 hours before adding sat. NH₄Cl (30 mL). The resulting mixture was then extracted with diethyl ether (3×30 mL). The organic layers were combined, washed with water (30 mL), brine (30 mL), dried over MgSO₄ and concentrated under vacuum. The oil was dried under high vacuum to afford the corresponding bromoalkyne as a pale-yellow oil and taken forward to the next step. ¹H NMR (500 MHZ, CDCl₃) 1.11 (m; no alkyne proton). 13C NMR (101 MHZ, CDCl₃)=83.50, 61.72, 18.49, 11.29.

(5,5-diethoxypenta-1,3-diyn-1-yl) triisopropylsilane (2): In a round-bottomed flask containing CuCl (4 mg, 0.38 mmol, 0.05 equiv) was added 30% aqueous n-BuNH₂ solution (0.5 M), creating a blue solution. The reaction was cooled to 0° C. A pinch of NH₂OH·HCl was added until, causing the reaction mixture became colourless. Propargylaldehyde diethyl acetal alkyne (0.99 mL, 7.59 mmol, 1.1 eq.) was added at 0° C. to the flask and the solution developed a slightly yellow colour. After 5 minutes, (Bromoethynyl)triisopropylsilane (2.18 g, 8.34 mmol, 1.0 eq.) was added drop wise at 0° C. The reaction mixture was allowed to warm to room temperature and monitored by TLC. Once the alkyne was consumed, the solution was exposed to air and diluted with brine (100 mL). The resulting mixture was extracted with DCM (3×10⁰ mL) and the pooled organic fractions were dried over MgSO₄, filtered and concentrated under reduced pressure. Silica gel column chromatography using a gradient of 100% pentanes to 5% EtOAc/95% pentanes gave diyne 2 as a yellow oil (2.57 g, 57% yield). ¹H NMR (500 MHZ, CDCl₃) 5.30 (s, 1H), 3.75 (dd, J=9.4, 7.1, 2H), 3.60 (dd, J=9.4, 7.1, 2H), 1.24 (t, J=7.1, 6H), 1.07 (m, J=1.0, 21H). ¹³C NMR (126 MHZ, CDCl₃=91.41, 88.43, 85.88, 71.31, 70.44, 61.23, 18.47, 15.01, 11.17. ESI LRMS m/z calculated for [C₁₈H₃₃O₂Si]⁺ [M+H₂O+H]⁺ 309.54, found 309.17.

2-(diethoxymethyl) tellurophene (3): To a round bottom flask, ground tellurium metal (0.323 g, 2.53 mmol, 1.2 equiv.) and sodium borohydride (0.384 g, 10.1 mmol, 4.8 equiv.) were added. The flask was purged with argon multiple times. Degassed water (10 mL) was added to the flask containing tellurium and sodium borohydride and set to stir at 50° C. under argon. Over an hour, the metallic suspension turned dark purple and eventually became a homogenous colourless solution. Simultaneously, a 20 mL scintillation vial charged with diyne 2 (0.650 g, 2.10 mmol, 1 eq.) was purged with argon. THF (10 mL) was added to the vial and the yellow solution was cooled to 0° C. using an ice-water bath. TBAF (1.0 M in THF, 8.43 mL, 8.43 mmol) was added dropwise to the vial and allowed to stir for approximately an hour. The diyne solution was warmed to room temperature and slowly concentrated using a rotatory evaporator. The crude mixture was diluted with Et₂O (30 mL) and brine (30 mL). The organic layer was separated and washed with brine twice (2×25 mL) and concentrated under reduced pressure without heating, to produce a deep red oil, which will decompose upon standing. The oil was taken up in degassed EtOH (10 mL) and purged with an argon balloon with sonication. This solution was added dropwise via syringe to the sodium hydrogen telluride solution. After 12 hours, TLC indicated diyne starting material was consumed and a new, UV-active spot had appeared. The flask was cooled to room temperature and then exposed to air for at least an hour. The crude mixture was filtered through a celite pad. A small volume of EtOH (˜5 mL) was used to wash the celite pad. The filtered orange-red solution was diluted with DCM (50 mL) and brine (50 mL) and extracted with DCM (5×30 mL). The separate organic layers were collected, dried over MgSO₄, filtered and concentrated under reduced pressure. Silica gel column chromatography was used to purify the tellurophene with a gradient of 100% pentanes to 10% EtOAc/90% pentanes afforded tellurophene 3 as a deep red-orange oil (52% yield). ¹H NMR (400 MHZ, CDCl₃)=8.89 (dt, J=6.8, 1.3, 1H), 7.81 (ddd, J=6.8, 4.0, 1.3, 1H), 7.71-7.65 (m, 1H), 5.62 (s, 1H), 3.75 (m, 2H), 3.69-3.57 (m, 2H), 1.27 (td, J=7.1, 1.3, 6H). ESI LRMS m/z calculated for [C₄H₃Te]− [M−C₅H₁₁O₂]⁻ 180.93, found 181.14.

2-tellurophenecarboxylaldehyde (4): A scintillation vial (20 mL) was charged with diethyl acetal protected tellurophene 3 (0.300 g, 1.06 mmol). THF (5 mL) was added and the reaction was set to stir. HCl (1.0 M, 5 mL) was added to the vial and the solution was allowed to stir for 6 hours at room temperature. The reaction mixture was transferred to a separatory funnel and diluted with DCM (20 mL) and brine (20 mL). The layers were separated and the aqueous layer was washed with DCM (3×20 mL). The DCM portions were combined, dried over MgSO₄ and filtered. Concentration under reduced pressure yielded the corresponding aldehyde 4 as a red liquid. ¹H NMR (400 MHZ, CDCl₃)=9.59-9.57 (m, 1H), 9.49 (d, J=6.7, 1H), 8.53 (dd, J=4.1, 1.3, 1H), 8.06 (dd, J=6.6, 4.1, 1H). ¹³C NMR (101 MHZ, CDCl₃)=187.82, 150.60, 147.45, 138.72, 138.41. ESI LRMS m/z calculated for [C₄H₃Te]− [M−CHO−H]− 180.93, found 181.14.

4-Demethylepipodophyllotoxin: In a round bottom flask (50 mL), a suspension of etoposide (0.200 g, 0.358 mmol) in 20% aqueous acetic acid (2.5 mL) was heated at 75° C. for 20 h. The resulting solution was concentrated to dryness under high vacuum. The resulting white crude material was dissolved in a 1:1 mix of DCM: MeOH and adsorbed onto silica, followed by purification using silica gel column chromatography with a gradient of 100% DCM to 20% MeOH/80% DCM. Hydrolyzed etoposide derivative was obtained as a white powder (66% yield). ¹H NMR (500 MHZ, CD₃OD)=6.98 (s, 1H), 6.50 (s, 1H), 6.26 (s, 2H), 5.94 (dd, J=7.2, 1.2, 2H), 5.10 (d, J=3.2, 1H), 4.55 (d, J=5.4, 1H), 4.54-4.45 (m, 2H), 4.32 (dd, J=8.7, 7.7, 1H), 3.94 (dd, J=11.8, 2.0, 1H), 3.70 (s, 6H), 3.68-3.65 (m, 1H), 3.49 (dd, J=14.0, 5.4, 1H), 3.38-3.19 (m, 3H; overlapping with CH3OH resonance), 2.94 (m, 1H). ESI LRMS m/z calculated for [C₂₇H₃₁O₁₃] [M+H] 563.52, found 563.18. 13C NMR (126 MHZ, CD₃OD)=176.65, 148.53, 147.10, 146.86, 134.30, 133.01, 130.69, 128.44, 110.14, 109.67, 107.90, 101.40, 100.39, 76.51, 73.62, 71.62, 70.39, 68.50, 61.62, 55.60, 43.69, 41.03, 37.90, 19.84.

Tellurium-labelled teniposide (1): In a 1 dram vial, zinc chloride (12 mg, 0.89 mmol, 2.0 equiv) and stir flea were added and flame-dried for 2 minutes. The flask was immediately placed under vacuum for at least 30 minutes. 4-Demethylepipodophyllotoxin (25 mg, 0.44 mmol, 1.0 equiv.) was quickly added and the vial was purged with argon numerous times. Aldehyde 4 (˜0.250 g) was added dropwise and the vial was carefully sonicated to ensure all solid materials were in suspension. The reaction was set to stir gently at room temperature for 16 hours. The solution was diluted with DCM (10 mL) and washed with brine (10 mL). The aqueous layer was separated and washed with DCM twice (10 mL). Together, the organic fractions were pooled, dried over MgSO₄ and concentrated. The residue was purified with silica gel column chromatography using gradient of 10% to 50% ethyl acetate in pentanes, affording 1 (16%) as a pale yellow waxy solid. ¹H NMR (500 MHZ, C₂D₆SO)=8.89 (dd, J=6.6, 1.4, 1H), 8.23 (s, 1H), 7.87-7.58 (m, 2H), 7.01 (s, 1H), 6.52 (s, 1H), 6.17 (s, 2H), 6.10-5.93 (m, 2H), 5.64 (s, 1H), 5.24 (s, 2H), 4.93 (d, J=3.4, 1H), 4.60 (d, J=7.6, 1H), 4.48 (d, J=5.4, 1H), 4.33-4.21 (m, 2H), 4.20 (dd, J=10.2, 4.8, 1H), 3.74 (t, J=10.1, 1H), 3.60 (s, 6H), 3.48-3.23 (m, 5H), 3.09 (t, J=8.0, 1H), 2.88 (dddd, J=13.7, 10.0, 8.2, 3.4, 1H). 13C NMR (126 MHz, C₂D₆SO)=175.18, 148.18, 147.71, 147.57, 146.59, 136.67, 135.50, 135.09, 133.27, 130.67, 129.31, 129.16, 110.35, 108.77, 101.78, 101.76, 101.73, 80.76, 74.88, 73.15, 72.13, 68.18, 68.09, 66.14, 55.50, 40.89. ESI HRMS m/z calculated for [C₃₂H₃₂O₁₃¹³⁰TeNa]⁺ [M+Na]⁺ 777.081, found 777.079.

### In Vitro Experiment

Top2 decatenation assay: The assay was performed according to manufacturer's protocol (TopoGEN, Inc.). The total reaction volume was 20 μL of assay buffer (120 mM KCl, 50 mM Tris-HCl, 10 mM MgCl2, 0.5 mM DTT, 0.5 mM ATP, and 30 μg/mL BSA) and 120 ng of kinetoplast DNA (kDNA) substrate. One unit of TopoII enzyme in was added to reaction microfuge tubes in the presence or absence of inhibitor. The reaction was incubated for 30 min at 37° C. The reaction was stopped by addition of 5 μL of stop buffer (5% sarkosyl, 0.025% bromophenolblue, and 50% glycerol). The samples were then analyzed using electrophoresis using a 1% agarose gel in Tris-borate-EDTA buffer with 0.5 pg/mL ethidium bromide stain. Gels were imaged using Syngene G: Box Gel Imager (Chemi-XT4 GENESys software with preset for ethidium bromide stained agarose gels). Band intensities were analyzed using ImageJ software.

WST-1 metabolic cytotoxicity assay: HL-60 cells (ATCC® CCL-240TM) were maintained in DMEM media with 2 mM L-glutamine (Gibco) supplemented with 20% calf serum and 100× dilution of penicillin-streptomycin solution (Gibco). Cells were maintained at 37° C. in a humidified atmosphere of 5% CO₂ in air. HL-60 cells (100 pL) were seeded into a 96 well clear at a density of 5×10⁵ cells/mL. Cells were treated with either DMSO or appropriate inhibitor from DMSO stocks. Dilutions were done carefully such that DMSO concentration did not exceed 1%. Cells were incubated for 24 h at 37° C. under a 5% CO₂ atmosphere. WST-1 reagent (10 pL; Roche Diagnostics, 05015944001) was added to each well and gently mixed by pipetting. Cells were incubated for 1 h at 37° C. under 5% CO₂ atmosphere. UV-vis absorbance measurements of each well at 450 nm was recorded using a TECAN Safire 2 plate reader. Data was background corrected against wells that contained cell growth media without cells and WST-1. Experiment was performed in triplicate.

IncuCyte ZOOM™ Cellular Proliferation assay: Human pancreatic ductal carcinoma (PANC-1) cell lines were purchased from ATCC (CRL-1469). The cells were cultured in Roswell Park Memorial Institute (RPMI) medium, supplemented with 10% FBS. Cell maintenance and experiments were performed in a humidified 37 C incubator with 5% CO₂. Cells were routinely tested for mycoplasma contamination. PANC-1 cells (5000) were seeded in a 96-well plate and incubated for 18h. Media was then removed and replaced with fresh media containing drug (0-25 pM). The cells were then transferred to IncuCyte ZOOM system (ESSEN BioScience, Ann Arbor, MI, USA) and live cell phase contrast images were obtained using a 10× objective lens. Cellular confluence was analyzed using IncuCyte ZOOM 2016B software.

pH2AX Blotting: PANC-1 cells (0.5×10⁶) cells were seeded in 60 mm plastic petri dishes (Corning Inc. NY) and incubated for 18 h. The spent medium is removed and replaced with fresh medium containing drug (1 pM) and incubated for either 24 or 48h. Control cells were treated with DMSO only. The cells were then lysed with RIPA buffer (25 mM Tris·HCl PH 7.6, 150 mM NaCl, 1% NP-40, 1% sodium deoxycholate, 0.1% SDS) (ThermoFisher Scientific) containing Halt Protease Inhibitor Cocktail (Cat. No. 78410) and Halt Phosphatase Inhibitor Cocktail (Cat. No. 78420) as per manufacturer's protocol. Extracted protein samples were subjected to electrophoresis using a Bolt 4-12% Bis-Tris, 1.0 mm gel (ThermoFisher Scientific) and immunoblotted as suggested. Antibodies (Abcam, Cambridge, MA) targeting the following proteins were used H2AX (S139) (ab11174, polyclonal) and β-tubulin (ab6046, polyclonal).

AlamarBlue assay: PANC-1 cells were seeded into black, clear-bottom 96-well assay plates (Corning; CLS3603) and incubated for 24 hours. The medium was replaced with 0.01-25 pM of teniposide or compound 1. After 72 hours exposure to drug, alamarBlue (Thermo Fisher Scientific; DAL1100) cell viability reagent was added and incubated for 4 hours at 37° C. Fluorescence intensity was measured using an excitation wavelength of 560 nm and an emission of 590 nm. Cell viability was calculated by normalizing to an untreated control.

CYTOF® labelling: For pre-saturation experiment, 3×10⁶ HL-60 cells (5×10⁵ cells/mL) were incubated with appropriate teniposide concentration for 2 hours at 37° C. in a humidified atmosphere of 5% CO₂ in air. Compound 1 was pipetted into the cell suspension and gently mixed. Cells were further incubated for 2 hours and then centrifuged for 6 min at 300×g, and the media was aspirated. For controls, HL-60 cells were treated with teniposide or compound 1 for 4 hours consecutively. For co-incubation experiments, cells were incubated with drug cocktail for 4 hours. Drug-treated cells were washed with media and then PBS. Cells were fixed with 3.7% formaldehyde (Sigma Aldrich F1635) diluted in PBS for 10 minutes. Fixed cells were centrifuged for 5 min at 800×g and washed with PBS. Then, cell pellets were stained with Ir-intercalator (1:1000 dilution in PBS) for 1 hour at room temperature. Cells were centrifuged at 800×g for 5 minutes, followed by 2 PBS washes and a final ddH₂O wash. Cell pellets were taken up in 10% EQ™ four-element calibration beads solution prepared in CAS and filtered into polystyrene tubes through 35 pm cell strainer caps. Samples were then injected into the CYTOF® Helios™ and analyzed.

### In-Vivo Experiments

Generation of murine PANC-1 xenograft model: All 6-week-old female NOD SCID mice (Charles River (Wilmington, MA) were maintained under specific pathogen-free (SPF) conditions at the Princess Margaret Cancer Centre (PMCC) facility. All animal experiments in this study were performed in accordance with the Animal Research Committee guidelines of the University Health Network institution. The subcutaneous PANC-1 xenograft mice were generated as described previously in Telox studies. The vehicle was prepared as follows: 30 mg benzyl alcohol, 60 mg N,N-dimethylacetamide, 500 mg purified Cremophor EL (Kolliphor EL), and 2.1 mL dehydrated alcohol were combined in a vial. The solution was topped with distilled water for a final volume of 5 mL. The pH of the clear solution is adjusted to pH ˜5 with maleic acid. For injections, drug was diluted with vehicle for a final concentration of 10 mg/mL and administered at 20 mg/kg by I.P. The mice were maintained in three groups; group 1 mice were injected with teniposide, group 2 received 1, and group 3 with normal saline. After 2 hours, mice were scarified, and tumors were extracted. Half of the tumor was fixed and carried forward for paraffin embedding while the remaining xenograft tissue was embedded in OCT (Tissue-Tek Sakura-Finetek and flash frozen in liquid nitrogen. Flash frozen tumor samples were stored at −80° C. Cryostat sections were cut (5 pm) using a microtome and mounted on microscope slides. These sections were stored at −80° C. until IMC™ analysis or histochemical staining. Sections were subjected to H&E staining to assess the morphology of the tissue and pH2AX IHC staining for visualizing pH2AX punctae. Adjacent sections stained for IMC™ analysis. Optical imaging of tissue sections: Tissue sections were fixed and blocked as previously described. Unconjugated rabbit anti-mouse H2AX (pSer139) antibody (Abcam, ab11174) and HRP conjugated anti-rabbit IgG were used for color development with 3,3-diaminobenzidine chromogen). Whole stained sections were then scanned using ScanScope AT2 (Aperio) at 20× magnification (˜0.5 pm/pixel) and viewed using ImageScope software.

IMC™ analysis: Frozen tissue sections (5 pm) were thawed from −80° C. to room temperature and directly subjected to IMC™ analysis on the HYPERION™ Imaging System (Fluidigm) without DNA staining or wash steps. To evaluate tellurium sensitivity of IMC™, serial dilutions of a tellurium standard solution (Sigma Aldrich Cat. No. 92027) were prepared in Trypan Blue. Volumes of 2 pL were arrayed on an air-dried 2% agarose coated slide microscopy slide for final concentrations ranging from 0-50 pM.

IMC™ data acquisition and analysis: Slides were ablated at 200 Hz using the HYPERION™ Imaging System (Fluidigm) and images were obtained as .txt files. Each image was unpacked into separate numpy arrays for each mass channel using the teimc package by Bassan and Nitz.²⁵ These numpy arrays were used to prepare raw images and subsequent analysis using the Numpy, pandas, Matplotlib, and Scikit-image libraries. For the tellurium standard curve, entire spots were ablated and the ¹²⁵Te counts across the entire cross-sectional area were totalled (¹²⁵Tetotal). For the blank spot ([Te]=0 μM), the average ¹²⁵Te intensity per pixel was calculated by dividing the total ¹²⁵Te signal by the number of pixels in the ablation cross-sectional area. This value represents the background signal per pixel in the 125 amu channel corresponding to the particular HYPERION™ Imaging System (¹²⁵Tebackground). To calculate contribution of this background across the tellurium-containing images, we performed the following correction for each concentration,

$${}_{}^{125} {Te}_{corrected}^{} ( i ) = {}_{}^{125} {Te}_{total}^{} ( i ) - ( {}_{}^{125} {Te}_{background}^{} \times area ( i ) )$$

The resulting series of ¹²⁵Tecorrected values were plotted against the total number of Te atoms calculated from the volume of standard solution dispensed. A linear regression model was fitted for the data points within the limit of linearity to calculate the number of tellurium atoms in the xenograft sections. Finally in order to calculate [Te] in tissue sections, the tissue volume was inferred by multiplying the cross-sectional area (1 mm×1 mm) by tissue section thickness (5 μm).

### Example 2: Tellurophene-Tagged Carfilzomib Analogue: Synthesis and Evaluation

Monitoring target engagement in vivo is a challenging multidimensional problem. Few methods are available to quantify engagement at the cellular level. The use of tellurium in combination with mass cytometry enabled engagement to be characterized at the cellular level in heterogenous samples.

L-2-tellurienylalanine (TePhe) has been demonstrated to act as a bio-isostere for phenylalanine (Phe). A TePhe containing version of the peptide drug Carfilzomib was synthesized. Carfilzomib is an irreversible inhibitor of the β5 chymotrypsin-like site of the proteasome and an FDA approved drug against multiple myeloma.

TePhe substituted Carfilzomib analogue (TeCar, compound of Formula II, 7b) was synthesized according to Scheme 2. The final peptide coupling of the epoxide warhead led to isomerization of the penultimate residue. This epimer (8b) was used as a control in the biological studies.

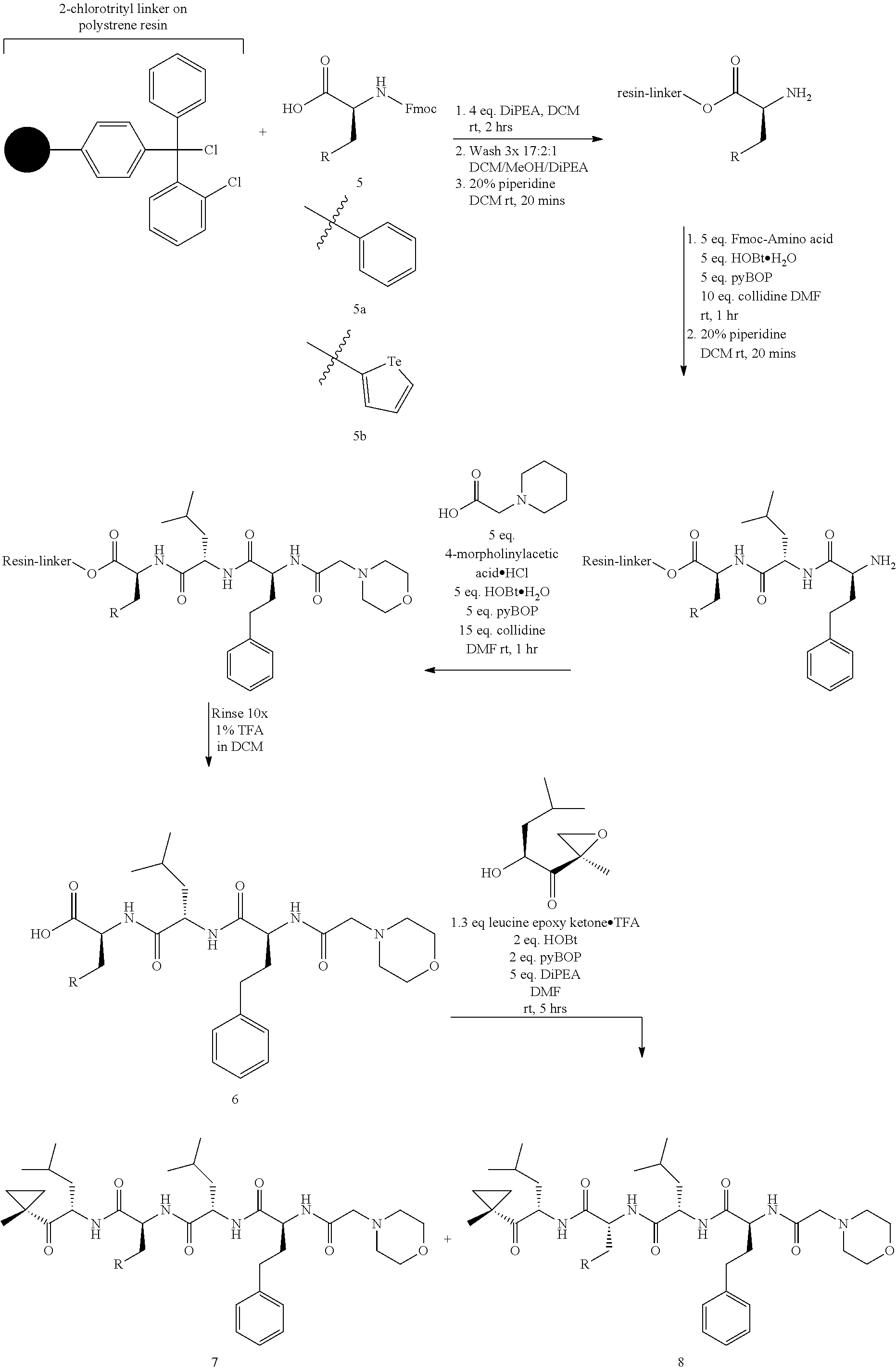

Scheme 2—Synthesis of TeCar Analogue 7b
(Compound of Formula II)

| Cpd | R | Name | Yield |
|---|---|---|---|
| 5a | 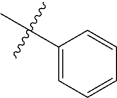 [SMILES: *c1ccccc1] | Fmoc-Phe- OH | |
| 5b | 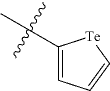 | Fmoc- TePhe-OH | |
| 6a | 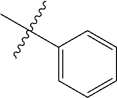 [SMILES: *c1ccccc1] | Carfilzomib int. | 57% |
| 6b | 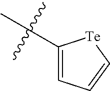 | TeCar int. | 89% |
| 7a 8a | 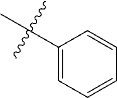 [SMILES: *c1ccccc1] | Carfilzomib | 35% |
| 7b 8b | 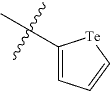 | TeCar | ~14% |

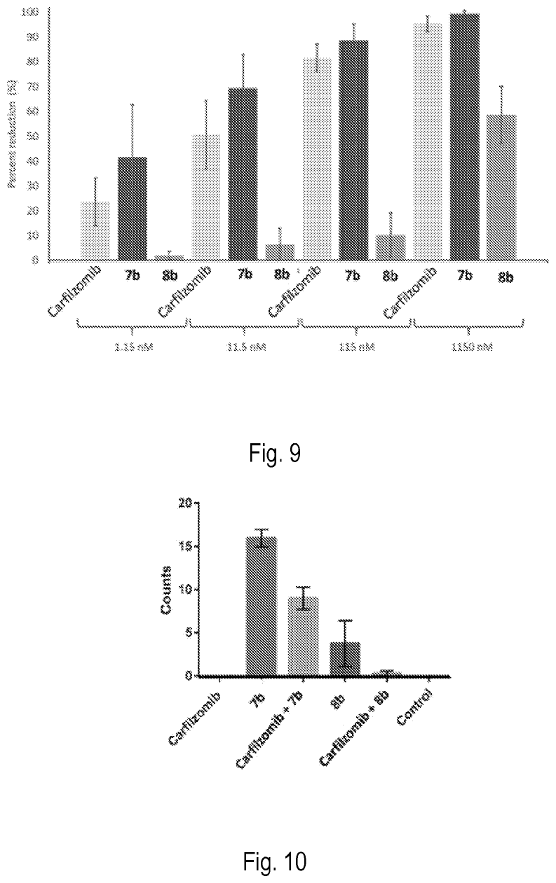

In vitro experiments with purified proteasome led to the reduction in activity expected with the covalent inhibitor. The activity observed was indistinguishable from Carfilzomib (FIG. 9).

In Jurkat cells treated with increasing concentrations of TeCar 7b, a concentration-dependent reduction in overall cell viability was observed (Table 1). When compared to Carfilzomib, similar concentrations of 7b (Te-Car) were needed to produce a 50% reduction in Jurkat cell viability.

TABLE 1
Viability Jurkat cells treated with Inhibitors

| Biol. Rep # | IC₅₀ Carfilzomib (nM) | IC₅₀ 7b (nM) |
|---|---|---|
| 1 | 9.127 | 9.481 |
| 2 | 9.992 | 9.869 |
| 3 | 9.412 | 9.777 |
| Average +/− SD | 9.510 +/− 0.441 | 9.709 +/− 0.293 |

Analyzing TeCar-treated Jurkat cells by mass cytometry showed activity dependent accumulation of tellurium within the cells. Co-dosing of cells with Te-Car 7b and Car lead to an approximately 50% reduction in Te labelling confirming the on-target labelling of modified compound (FIG. 10). This evidence supports that TeCar can be used to monitor Carfilzomib's target engagement in a more complex and disease-relevant samples.

### Methods

### Synthesis General Methods

All reagents were purchased from Sigma-Aldrich unless otherwise specified. Solvents were degassed by bubbling with N_{2(g)} prior to solid-phase peptide synthesis (SPPS) reactions, with Ar_{(g)} prior to in-solution reactions, or with He_{(g)} prior to reverse-phase high-performance liquid chromatography (RP-HPLC) purifications. Resin swelling and SPPS were carried out in fritted polypropylene chromatography columns purchased from Bio-rad. Flash column chromatography was performed using SILIAFLASH™ P60 from Silicycle. Solvents were evaporated using a BUCHI™ rotary evaporator. RP-HPLC was performed using a Waters 1525 binary HPLC pump with a Waters XBRIDGET Prep BEH130 C₁₈ 10×250 mm column, coupled to a Waters 2487 dual λ absorbance detector. Lyophilization was performed on a THERMO MODULYO™ Freeze dryer. Low-resolution and high-resolution mass spectra were acquired using a Bruker AUTOFLEX SPEED™ matrix-assisted laser desorption ionization time-of-flight mass spectrometer (MALDI-TOF-MS), or an Agilent 6538 Q-TOF mass spectrometer coupled to an electrospray ionization (ESI) source, respectively. Nuclear magnetic resonance (NMR) spectra were acquired on either a 500 MHz Agilent DD2 spectrometer with an XSENS™ C13 Cold Probe or on a 700 MHz Agilent DD2 spectrometer.

Synthesis of 5b was carried out as per 10. Once isolated via crystallization and verified for adequate purity using ¹H NMR (500 MHz in CDCl₃), 5b was used in the following SPPS steps without further purification.

### Synthesis, Purification, and Characterization of 6a and 6b

6a and 6b were synthesized using SPPS. Briefly, Fmoc-L-Phe-OH (5a) (1 eq., 77 mg) or Fmoc-L-TePhe-OH (5b) (1.25 eq, 100 mg) was dissolved in anhydrous (anh.) DCM (2-2.5 mL). To the solution, diisopropylethylamine (DiPEA) (4 eq, 110-139 μL). was added, and the solution was added onto dry 2-chlorotrityl resin (163-200 mg, 1 mmol/g) in a fritted polypropylene tube (10 mL). The tube was then allowed to invert at room temperature (rt) for 2 hours. The reaction mixture was evacuated from the column using vacuum filtration, and the resin was washed three times by bubbling the resin in a capping mixture (17:2:1 anh. DCM/MeOH/DiPEA) with N_{2(g)} for one minute before being evacuated using vacuum filtration. The resin was then washed repeatedly with alternating DCM and DMF in a similar fashion. The resin was then allowed to invert in a 20% piperidine solution (3 mL) in anh. DMF at rt for 20 mins to deprotect the Fmoc group. Again, the solvent was evacuated and the resin washed repeatedly with DMF and DCM. Next, Fmoc-L-Leu-OH (5 eq.) was coupled using HOBt·H₂O (5 eq.), pyBOP (Fluka) (5 eq.), and of collidine (10 eq.) in anh. DMF ([resin]=0.04M) at rt for 1 hr, the resin was then washed and the Fmoc group deprotected as above. These steps were then repeated using Fmoc-L-homophenylalanine-OH (Alfa Aesar) (5 eq.), and then with 4-morpholinylacetic acid HCl salt (Alfa Aesar) (5 eq.) with extra collidine (15 eq.). For each of these three couplings, the carboxylic acid was first allowed to be activated with HOBt/pyBOP in the presence of the collidine for 8 mins at rt before being added onto the resin.

The resin was then washed 10× with 1% trifluoroacetic acid (TFA) in anh. DCM (1 mL) and each wash fraction was collected into a round bottom flask. The solution was evaporated and re-diluted many times with toluene, before being allowed to fully dry under vacuum to yield a yellow oil.

The resulting oil was then assessed with MALDI-TOF-MS to confirm the presence of the desired peptide (6a or 6b). The peptide crude dissolved in 1:1 MQ H₂O/ACN was co-crystallized with α-cyano-4-hydroxycinnamic acid (CHCA) matrix. A 567 m/z peak ([M+H]) confirmed the presence of 6a, while peaks at 667, 669, 671 and 689, 691, 693 m/z ([M+H] and [M+Na] of the 3 most abundant Te isotope species, respectively) confirmed the presence of 6b.

6a and 6b were then purified using flash silica chromatography or RP-HPLC.

For flash silica chromatography, a solvent system of 9:1 DCM/MeOH+1% AcOH was used, and product elution was evaluated using thin layer chromatography and visualized using a short-wave UV lamp and KMnO₄ stain.

For RP-HPLC, a 90 min gradient of solvent A (MQ H₂O+0.1% TFA) from 98% to 2% in solvent B (ACN+0.1% TFA) was used, and product elution was evaluated by monitoring at 213 and 254 nm (6a) or 213 and 280 nm (6b) wavelengths.

Like fractions from either method were combined, concentrated, and assessed for purity using MS. Similar levels of purity were obtained with both methods, with flash silica chromatography being more expedient.

The structure of 6b was later analyzed using ¹H NMR acquired at 500 MHz at a concentration of approximately 0.02M in D₆-DMSO. ¹H NMR (700 MHZ, dmso) δ 10.21 (bs, 1H), 8.83 (bs, 1H), 8.61 (dd, J=6.9, 1.3 Hz, 1H), 8.32 (d, J=8.0 Hz, 1H), 8.18 (d, J=8.5 Hz, 1H), 7.50 (dd, J=6.9, 3.8 Hz, 1H), 7.40 (dq, J=3.8, 1.3 Hz, 1H), 7.33-7.26 (m, 2H), 7.22-7.14 (m, 3H), 4.43 (dtd, J=11.4, 8.3, 5.5 Hz, 2H), 4.33 (ddd, J=9.1, 8.0, 4.4 Hz, 1H), 3.98 (bs, 2H), 3.90 (bs, 2H), 3.77 (bs, 3H), 3.34 (ddd, J=15.0, 4.4, 1.1 Hz, 1H), 3.18 (bs, 4H), 3.11 (ddd, J=15.0, 9.1, 1.3 Hz, 1H), 2.61 (ddd, J=13.5, 11.3, 5.2 Hz, 1H), 2.55 (ddd, J=13.7, 11.0, 5.9 Hz, 1H), 1.95 (ddt, J=13.4, 11.0, 5.4 Hz, 1H), 1.89-1.76 (m, 1H), 1.61 (ddt, J=14.8, 13.0, 6.4 Hz. 1H), 1.52-1.40 (m, 2H), 0.87 (dd, J=26.9, 6.6 Hz, 6H). HRMS: calculated m/z for C₂₉H₄₀N₄O₆¹³⁰Te (M+H)+ 671.5636; found 671.21.

In-Solution Synthesis of 7a/8a (Carfilzomib and Epimer Thereof) and 7b/8b (Te-Carfilzomib Analogue and Epimer Thereof)

1 eq. of either 6a (30 mg) or 6b (45 mg) (both white solids) was dissolved in degassed anh. DMF (1.5 mL) to which a solution of HOBt anh. (2 eq.), pyBOP (2 eq.), and DiPEA (5 eq.) in degassed anh. DMF (2 mL) was then added. The reaction was allowed to stir at rt under Ar_{(g)} for five minutes before (2S)-2-amino-4-methyl-1-[(2R)-2-methyloxiranyl]-1-pentanone trifluoroacetate (1.3 eq.) (Ontario Chemicals Inc.) dissolved in degassed anh. DMF (1.5 mL) was added. The final concentration of peptide 6a or 6b was approximately 0.01 M. The reaction was stirred at rt under Ar_{(g)} for up to 5 hrs. The reaction was monitored hourly using MALDI-TOF-MS, using the same parameters as described previously. Once the complete loss in starting material was observed, the reaction was taken into ethyl acetate (10 mL) and washed twice using deionized water (10 mL×2). The ethyl acetate layer was then washed with an equal volume of brine, dried over powdered MgSO₄₍ₛ₎, filtered, and the solvent evaporated to yield a crude yellow oil that was then further dried under high vacuum to yield a yellow oil with white crystals. The crude was stored at −20° C. until purification.

Purification and Characterization of 7a/8a and 7b/8b

The crude was dissolved in minimal volume of ACN (˜2 mL) and aliquoted into fractions (100 μL). Each fraction was diluted with MQ™ H₂O (˜0.9 mL) and filtered using a 0.22 μm syringe prior to injection into the RP-HPLC. The solvent system used was 0.1% TFA in MQ H₂O for solvent A and 0.1% TFA in ACN for solvent B. The following gradient method was used:
0-10 min hold 98% A
10-40 min 98%-50% A
40-60 min hold 50% A
60-100 min 50-2% A
100-110 min hold 2% A

Peaks were monitored at 213 nm and 254 nm (7a/8a) or 213 nm and 280 nm (7b/8b). Only peaks with both 213 nm and either 254 or 280 nm character with at least A₂₁₃≥0.5 were collected and assessed using MALDI-TOF-MS, using the parameters specified above. The peaks which eluted at ˜60 mins (7a or 8a) and ˜64 mins (7b or 8b) were found to contain the masses consistent with the desired product.

7b: ¹H NMR (700 MHZ, dmso) δ 8.60 (d, J=6.8 Hz, 1H), 8.24 (s, 1H), 8.13 (d, J=8.3 Hz, 2H), 7.50 (dd, J=6.9, 3.8 Hz, 1H), 7.37 (d, J=5.0 Hz, 1H), 7.28 (t, J=7.6 Hz, 2H), 7.18 (dd, J=16.5, 7.7 Hz, 3H), 4.37 (s, 4H), 4.03 (bs, 1H), 3.91 (bs, 1H), 3.74 (bs, 3H), 3.22 (dd, J=15.3, 3.7 Hz, 3H), 3.14 (d, J=5.2 Hz, 2H), 3.00-2.95 (m, 3H), 2.53 (bs, 4H), 1.94 (s, 1H), 1.82 (s, 1H), 1.62 (s, 1H), 1.57 (s, 1H), 1.42 (d, J=7.4 Hz, 1H), 1.40 (s, 3H), 1.37-1.32 (m, 1H), 1.31-1.26 (m, 1H), 1.24 (bs, 1H), 0.92-0.85 (m, 6H), 0.82 (t, J=7.0 Hz, 6H). HRMS: calculated m/z for C₃₈H₅₅N₅O₇¹³⁰Te (M+H)⁺ 824.7845; found 824.32.

8b: ¹H NMR (700 MHZ, dmso) δ 8.69 (dd, J=6.9, 1.3 Hz, 1H), 8.25 (bs, 1H), 8.18 (bs, 1H), 8.12 (d, J=8.0 Hz, 1H), 7.49 (dd, J=6.9, 3.8 Hz, 1H), 7.36 (dd, J=3.8, 1.3 Hz, 1H), 7.27 (t, J=7.6 Hz, 2H), 7.21-7.14 (m, 3H), 4.44 (td, J=9.0, 4.6 Hz, 1H), 4.38 (dtd, J=21.8, 8.1, 4.3 Hz, 2H), 4.30 (bs, 1H), 3.88 (bs, 2H), 3.74 (bs, 3H), 3.20 (dd, J=14.2, 4.4 Hz, 3H), 3.12 (d, J=5.3 Hz, 2H), 2.99 (dd, J=14.5, 9.6 Hz, 1H), 2.91 (d, J=5.2 Hz, 1H), 2.59-2.50 (m, 2H), 1.92 (ddt, J=13.5, 10.9, 5.5 Hz, 1H), 1.80 (dddd, J=13.7, 10.9, 8.7, 5.3 Hz, 1H), 1.54 (dtd, J=9.5, 6.8, 4.8 Hz, 1H), 1.39 (d, J=6.7 Hz, 1H), 1.37 (s, 3H), 1.33 (dd, J=9.5, 3.6 Hz, 1H), 1.23 (bs, 1H), 1.29 (t, J=7.3 Hz, 3H), 0.84 (d, J=6.6 Hz, 3H), 0.79 (d, J=6.6 Hz, 6H), 0.77 (d, J=6.5 Hz, 3H). HRMS: calculated m/z for C₃₈H₅₅N₅O₇¹³⁰Te (M+H)⁺ 824.7845; found 824.32.

Subsequent runs had consistent and reproducible HPLC spectra, therefore latter HPLC runs were stopped at the 90 min mark and the column flushed for 10 mins with 100% B before starting the following purification.

As both Carfilzomib (“a”) and Te-Carfilzomib (“b”) compounds had two resolved peaks elute with the correct masses, they were kept separate, and the like fractions were termed Carfilzomib peak 1 (7a) and peak 2 (8a) and Te-Carfilzomib (TeCar) peak 1 (7b) and 2 (8b). All four combined fractions were lyophilized to yield a fluffy white powder and assess using ESI-MS to further confirm mass and purity.

7b and 8b were also assessing using 1H and ¹³C NMR (1D, gCOSY, and HSQC) to confirm that the fractions were epimers. These NMRs were acquired on the 700 MHz instrument in D₆-DMSO at sample volumes of 190-200 μL, due to low amounts of products isolated following HPLC.

7a and 8a were not assessed using NMR due to low combined yield isolated from the HPLC. Carfilzomib (Focus Biomolecules) was later purchased for further use in biological assays and for comparison purposes using NMR.

Stock solutions of 7b (232 uM), 8b (384 uM), and the purchased Carfilzomib (8800 μM) were made in DMSO and stored at −80° C. until needed. The concentration of the Carfilzomib stock was determined based off the mass weighed, while concentrations of the 7b and 8b solutions were confirmed using amino acid analysis, utilizing the Carfilzomib stock solution as a standard to confirm accuracy.

¹H NMRs acquired up to 8 months following storage at −80° C. confirmed no degradation occurred under these storage conditions.

### Biology General Methods

Purified human 26S proteasome was purchased from Novus™ biologicals. The β5 site activity probe, succinate-leucine-leucine-valine-tyrosine-7-amino-4-methylcoumarin (Succ-LLVY-AMC) was purchased from Enzo Life Sciences™, and 7-amino-4-methyl coumarin (AMC) from Sigma-Aldrich™. WST-1 cell viability reagent was purchased from Roche™. 96-well plates were purchased from Corning or Starstedt and all 96-well plate measurements were taken on a CLARIOSTAR® plate reader. Data was processed using Microsoft Excel and GraphPad Prism.

Jurkat cells (CRL-2899) were purchased from ATCC were maintained in Rosewell Park Memorial Institute (RPMI) media supplemented with 10% fetal bovine serum (FBS) and 1% penicillin/streptomycin in a humidified 37° C. incubator with 5% CO_{2 (g)}, and cultured as per ATCC guidelines in culture flasks purchased from Starstedt.

Cell-ID™ Intercalator-Ir was purchased from Fluidigm. Cytometry time-of-flight (CyTOF) measurements were recorded on a CYTOF2@ instrument. CyTOF data was processed and analyzed using FlowJo and GraphPad Prism software.

IN-Vitro Binding Assay with Purified Proteasome

To a 96-well plate 26S proteasome (2 nM, 25 uL) and varying concentrations of Carfilzomib, 7b, or 8b (1-100 nM, 25 uL) or blank control (1% v/v DMSO, 25 uL) in buffer (50 mM Tris-HCl buffer+1 mM DTT+5 mM MgCl₂+40 mM KCl+2 mM ATP+0.5 mg/mL BSA) were mixed, and allowed to incubate at room temperature for 15 min. Substrate peptide Suc-LLVY-AMC (200 μM, 50 μL) in buffer was then added to initiate the enzymatic reaction. The plate was placed in an incubator set to 37° C. for the remainder of the experiment, except when removed to take fluorescent measurements. The final well volume was 100 μL, and final concentrations of all reactants immediately following addition of substrate were: 0.5 nM (1 ug/mL) proteasome, 0.25-25 nM inhibitor, and 100 μM Succ-LLVY-AMC substrate.

The following controls were also tested as part of this experiment, in addition to a blank control ([inhibitor]=0 nM). Autohydrolysis of the substrate was assessed by incubating the substrate in buffer only (100 μM). Any chemical interactions between 7b or 8b and the substrate were assessed by incubating either drug (50 nM, 50 uL) with substrate (200 μM, 50 uL) in buffer. The substrate for each of these controls was added to the respective well at the same time as the experimental wells described above.

A standard curve of AMC (0-5 μM, 100 uL) was also prepared and ran alongside the assay. The standards were plated during the last 5 mins of the 15 min incubation, ie. immediately before the addition of substrate to the experimental wells.

Fluorescent measurements were then recorded by a microplate reader at λₑₓ of 350 nm and λₑₘ of 440 nm at time “zero” (˜3 mins following addition of the substrate), and then once every 15 mins for 75 mins total.

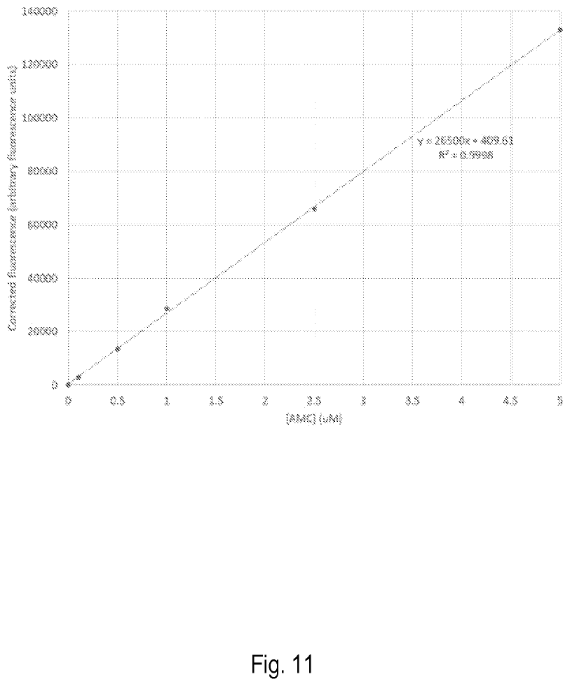

The standard curve served to prove the linear time dependence of the [AMC] in solution and its corresponding fluorescence reading over the range of the experiment. (See FIG. 11)

Each experimental and control well was corrected for autohydrolysis by subtracting out the fluorescence value for the autohydrolysis control well at the corresponding time point, before being analyzed further.

Apparent initial velocity (vₒ) of the uninhibited enzyme was first determined by plotting the average of the corrected fluorescence readings (FU, arbitrary fluorescence units) against time such that:

$$FU = {v}_{o}^{*} t + constant$$

where the slope of the plot was taken as the vₒ (uM substrate/min).

The vₒ kinetic parameter defining the remaining active enzyme in each well following dosing with either inhibitor over the concentration range of 1.15 nM-1.15 μM was determined similarly. The loss in vₒ (due to the decreased [E]ₜₒₜₐₗ and thus [E]_{active} in solution) was then compared to the uninhibited enzyme in the form of percent decrease, calculated using:

$$Percent decrease {v}_{o} = ( {v}_{o ( uninhibited )} - {v}_{o ( inhibited )} ) / {v}_{o ( uninhibited )} * 100 \%$$

This assay was repeated a total of three times. The averaged percent changes between these three biological replicates is shown in FIG. 10.

### Cell Viability in Jurkat Cells

Jurkat cells, at a concentration of approximately 12 500 cells/mL, were seeded in a 96 well plate in complete media in dosed with increasing concentrations (0-200 nM) of Carfilzomib or 7b (100 ul final volume) and left to incubate for 44 hours. Each concentration was repeated in triplicate. 10 uL of WST-1 was then added to each well, and the plate was allowed to incubate for 2 hours before the absorbance at 440 nm was measured. Absorbance signal in 100 uL complete media+10 uL WST-1 was first subtracted from each well before plotting the absorbance signal vs log (concentration). The concentration needed to see a 50% reduction in absorbance signal was then determined using GraphPad Prism software. These values are displayed in Table 1.

7b and 8b Incorporation into Living Jurkat Cells with or without Carfilzomib

Jurkat cells were seeded in 75 mm² plastic culture flasks at a concentration of approximately 5 million cells/mL for 24 hours prior to dosing. The cells were dosed with either Carfilzomib, 7b, or 8b at a final concentration of 500 nM, or a combination of Carfilzomib/7b (500 nM each), Carfilzomib/8b (500 nM each), or a DMSO control (final percentage of 0.5%) in complete media for 1 hour.

The media was then immediately removed, and the cells resuspended in 3 mL of cold phosphate-buffered saline (PBS), before being aliquoted into three 1 mL aliquots per each dosing regime. The samples were then prepped for CYTOF® analysis.

Briefly, each sample aliquot was washed three times with cold PBS (1 mL×3). The cells were then fixed, permeabilized, and stained with Cell-ID™ Intercalator-Ir stain in PBS (1 mL) for 1 hour at rt. The cell pellets were then washed two to three times with cold PBS (1 mL×2-3), then once with cold MQ H₂O (1 mL). The pellets were then stored overnight at 4° C.

CYTOF® analysis was conducted the following day. The cell pellets were resuspended in 250-500 ul of a bead solution in PBS and filtered immediately prior to injection into the instrument. Approximately 40 000 events were collected for each sample. The average ¹²⁸Te signal in the population of cells (events positive for 191 Ir and 193Ir signal) for each sample was determined.

### Exemplary Systems

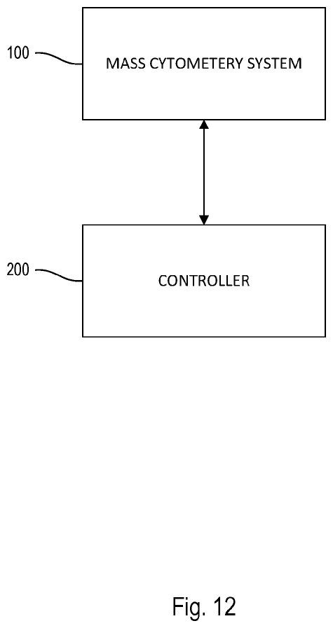

Referring now to FIG. 12, in some embodiments, a system for imaging a tissue or a cell includes an imaging mass cytometry system 100 and a controller (e.g., a computer system) 200 that is connected to and in communication with the imaging mass cytometry system 100 via a wired or wireless connection. As will be discussed in further detail herein, the controller 100 controls operation of the imaging mass cytometry system 100, receives an image from the imaging mass cytometry system 100, and display the received image.

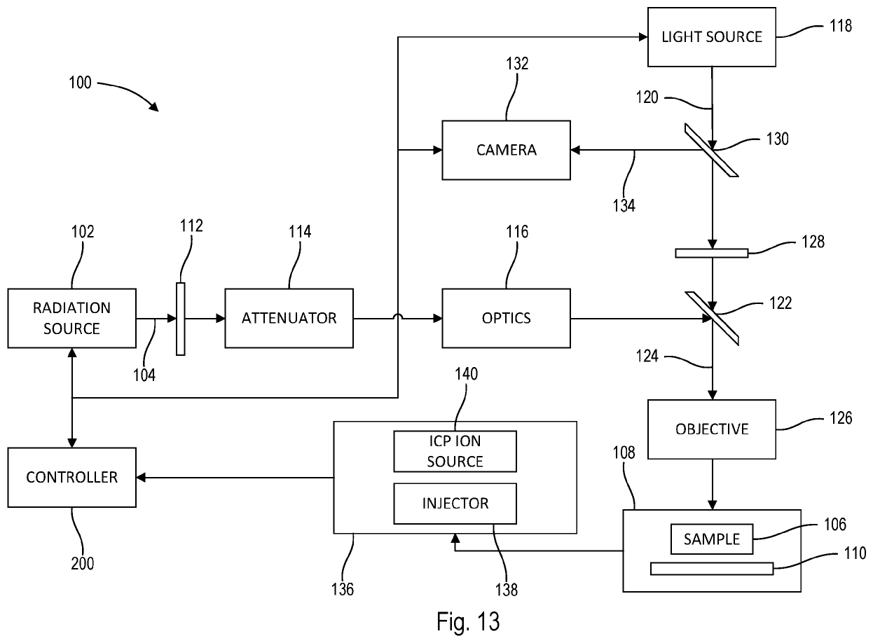

FIG. 13 depicts an exemplary imaging mass cytometry system 100 according to some embodiments of the disclosure. The system 100 includes a radiation source (e.g., a UV laser, femtosecond lasers, excimer lasers, etc.) that is configured to emit radiation (also referred to as “imaging light”) along a first pathway 104 towards a sample 106 that is positioned within a cell (e.g., a flow cell) 108. In some embodiments, the sample 106 may be positioned on a movable stage (e.g., an XYZ stage) 110 that is disposed within the cell 108.

The radiation source 102 is configured to transmit radiation for ablating and/or fluorescing the sample 106. In some embodiments, wherein the radiation source 102 is a UV laser, the radiation source 102 may operate at a wavelength of 213 nm. The irradiation of various spot sizes can be accomplished using a mechanically controlled aperture or an array of interchangeable apertures and/or an objective (e.g., along beam path 104) with proper magnification to establish the spot size or alternatively multiple laser shots can be scanned across the ablation area corresponding to one pixel by rapidly dithering the optics.

The system 100 may further include a shutter (e.g., a rastering shutter) 112 disposed between the radiation source 102 and the sample 106 along the pathway path 104. The shutter 112 provides energy stability to the system 100 by allowing continuous operation of the radiation source 102 while turning off delivery to the sample 106 during movement of the stage 110. The shutter 112 may also act as a safety feature which is activated when a safety interlock has been triggered in the system 100.

The system 100 also includes an attenuator 114, beam shaping optics 116. The attenuator 114 is disposed between the shutter 112 and the sample 106 along the pathway path 104. The attenuator 114 provides the ability to vary an energy of the radiation emitted by the radiation source 102 for accurate ablation conditions for a given sample 106. In some embodiments, the attenuator 114 operates based on polarization rotation and a polarizer filtering the radiation. The optics 116 are configured to shape the emitted radiation to produce a focused spot that is directed to the sample 106. The optics 116 may include one or more objectives and/or apertures for focusing the emitted radiation.

The system 100 further includes a light source (e.g., an LED) 118 that is configured to emit light along a second pathway 120 toward the sample 106. The system 100 also includes a first beam splitter (e.g., a dichroic mirror, a half-mirror, etc.) 122 positioned along the first pathway 104 between the optics 116 and the sample 106 and along the second pathway between the light source 118 and the sample 106. The first beam splitter 122 is configured to direct radiation received along the first beam pathway 104 and light emitted by the light source 118 along the second pathway 120 toward the sample 106 along a third pathway 124. The system 100 also includes a microscope objective 126 positioned along the third pathway 124 between the first beam splitter 122 and the sample 106. The microscope objective 126 focuses radiation from the radiation source 102 and light from the light source 118 onto the sample 106. Accordingly, in some embodiments, the system 100 may have a common focusing path for imaging light emitted by the radiation source 102 and light emitted by the light source 118.

The system 100 also includes a tube lens 128, a second beam splitter 130, and a camera (e.g., a charged coupled device image sensor based (CCD) camera, active pixel sensor based camera, etc.) 132. The tube lens 128 is positioned between the first beam splitter 122 and the second beam splitter 130 along the second pathway 120. The tube lens allows for the focusing of an optical image onto the camera 132. In some embodiments, the system 100 may employ an infinite conjugation setup using tube lens 128. This setup may reduce some types of aberrations and may also improve the quality of the image on the camera 132. The second beam splitter 130 may include, but is not limited to, a half mirror.

The second beam splitter 130 is positioned between the tube lens 128 and the light source 118 along the second pathway 120. The beam splitter directs light reflected off of the sample 106 towards the camera 132 along a fourth pathway 134.

The system 100 further includes a mass cytometer 136 that is coupled to the cell 108. The mass cytometer includes an injector 138, an inductively coupled plasma (ICP) ion source 140, and a detector 142. The injector 138 is configured to receive an ablated sample 106 from the chamber 108 and transition the ablated sample 106 to the ICP ion source 140. The ICP ion source 140 receives the ablated sample 106 and generates a plurality of ions. The detector (e.g., a time-of-flight (TOF) analyzer) 142 is positioned downstream from the ICP ion source 140 and receives the plurality of ions. The detector 142 provides a mass analysis of the ions that have exited the ICP ion source 140 according to the ions' mass to charge ration (m/z).

The image provided by the 132 and the mass analysis provided by the detector 142 may be output to the controller 200. The controller 200 may output the image and the mass analysis to a display. This output may allow a user to visually determine the location and level of a given molecule (e.g., an analogue of a small molecule as discussed herein) within the sample 106.

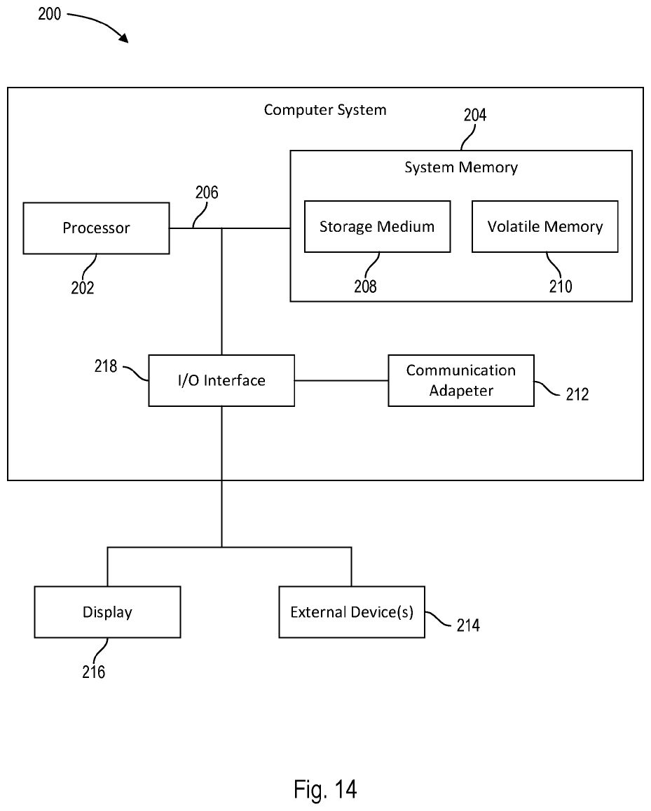

Referring now to FIG. 14, a computer system 200 is shown in accordance with an exemplary embodiment. The computer system 200 may serve as any computer system disclosed herein (e.g., the controller 200). As used herein a computer system (or device) is any system/device capable of receiving, processing, and/or sending data. Computer systems include, but are not limited to, microprocessor-based systems, personal computers, servers, hand-held computing devices, tablets, smartphones, multiprocessor-based systems, mainframe computer systems and the like.

As shown in FIG. 14, the computer system 200 includes one or more processors or processing units 202, a system memory 204, and a bus 206 that couples the various components of the computer system 200 including the system memory 204 to the processor 202. The system memory 204 includes a computer readable storage medium 208 and volatile memory 210 (e.g., Random Access Memory, cache, etc.). As used herein, a computer readable storage medium includes any media that is capable of storing computer readable; program instructions and is accessible by a processor. The computer readable storage medium 208 includes non-volatile and non-transitory storage media (e.g., flash memory, read only memory (ROM), hard disk drives, etc.). Computer program instructions as described herein include program modules (e.g., routines, programs, objects, components, logic, data structures, etc.) that are executable by a processor. Furthermore, computer readable program instructions, when executed by a processor, can direct a computer system to function in a particular manner such that a computer readable storage medium comprises an article of manufacture. Specifically, the computer readable program instructions when executed by a processor can create a means for carrying out at least a portion of the steps of the methods disclosed herein.

The bus 206 may be one or more of any type of bus structure capable of transmitting data between components of the computer system 200 (e.g., a memory bus, a memory controller, a peripheral bus, an accelerated graphics port, etc.).

The computer system 200 may further include a communication adapter 212 which allows the computer system 200 to communicate with one or more other computer systems/devices via one or more communication protocols (e.g., Wi-Fi, BTLE, etc.) and in some embodiments may allow the computer system 200 to communicate with one or more other computer systems/devices over one or more networks (e.g., a local area network (LAN), a wide area network (WAN), a public network (the Internet), etc.).

In some embodiments, the computer system 200 may be connected to one or more external devices 214 and a display 216. As used herein, an external device includes any device that allows a user to interact with a computer system (e.g., mouse, keyboard, touch screen, etc.). An external device 214 and the display 216 may be in communication with the processor 202 and the system memory 204 via an Input/Output (I/O) interface 218.

The display 216 may display a graphical user interface (GUI) that may include a plurality of selectable icons and/or editable fields. A user may use an external device 214 (e.g., a mouse) to select one or more icons and/or edit one or more editable fields. Selecting an icon and/or editing a field may cause the processor 202 to execute computer readable program instructions stored in the computer readable storage medium 208. In one example, a user may use an external device 214 to interact with the computer system 200 and cause the processor 202 to execute computer readable program instructions relating to at least a portion of the steps of the methods disclosed herein.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

1. A method of detecting a small molecule compound within a sample, the method comprising the steps of: providing either a subject or a cell/tissue culture specimen;
providing a small molecule analogue of a small molecule compound, wherein the small molecule analogue has a structure of Formula I or II: 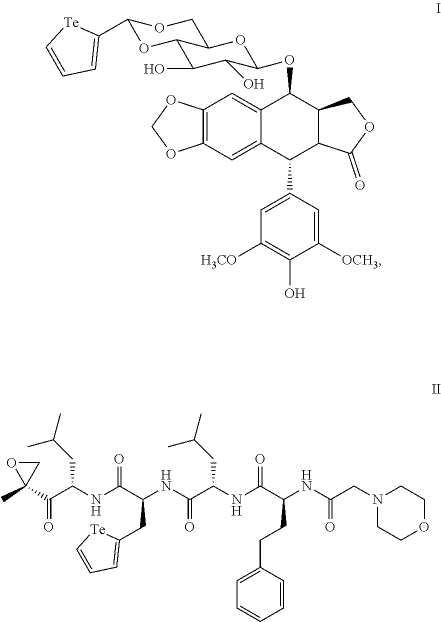
or a salt or solvate thereof,
administering the tellurophene small molecule analogue to the subject or cell/tissue culture specimen;
providing a sample which is taken either from the subject or from the cell/tissue culture specimen after administration of the tellurophene small molecule analogue;
performing mass spectrometry on the sample to determine a level of the tellurium atom present in the sample,
wherein the level of the tellurium atom corresponds to the level of the tellurophene small molecule analogue, and detection of the tellurophene small molecule analogue is indicative of detection of the small molecule compound in the sample.

2-40. (canceled)

41. A compound of Formula I 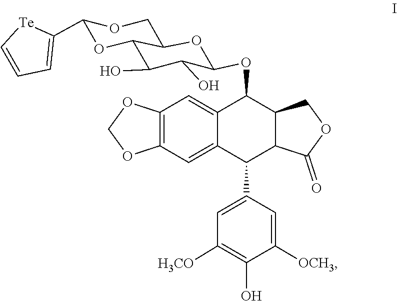
or a salt or solvate thereof.

42. The compound of claim 41, wherein the Te atom is an isotope, optionally the Te atom is selected from ¹²⁰Te, ¹²²Te, ¹²³Te, ¹²⁴Te, ¹²⁵Te, ¹²⁶Te, ¹²⁸Te, ¹³⁰Te, and combinations thereof.

43. A composition comprising the compound of claim 41 or a salt or solvate thereof, and a carrier or excipient.

44. A compound of Formula II 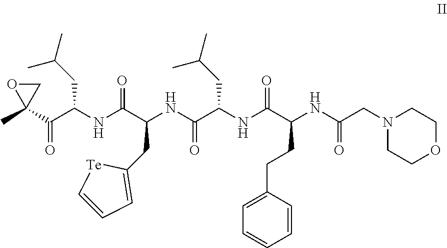
or a salt or solvate thereof.

45. The compound of claim 44, wherein the Te atom is an isotope, optionally the Te atom is selected from ¹²⁰Te, ¹²²Te, ¹²³Te, ¹²⁴Te, ¹²⁵Te, ¹²⁶Te, ¹²⁸Te, ¹³⁰Te, and combinations thereof.

46. A composition comprising the compound of claim 44 or a salt or solvate thereof, and a carrier or excipient.

47.-57. (canceled)

58. A kit for mass spectrometry analysis comprising a tellurophene analogue of teniposide of Formula I 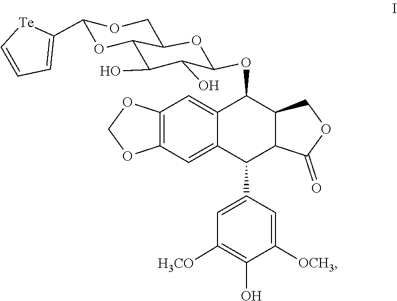
or a salt or solvate thereof,
or a tellurophene analogue of carfilzomib of Formula II 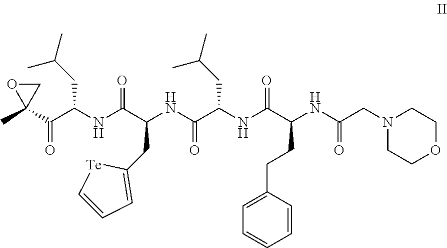
or a salt or solvate thereof, and
a tellurium standard or a plurality of tellurium standards.

59. The kit of claim 58, wherein the mass spectrometry analysis is mass cytometry analysis.

60. The kit of claim 58, wherein the tellurium in the analogue and the tellurium in the standards are selected from ¹²⁰Te, ¹²²Te, ¹²³Te, ¹²⁴Te, ¹²⁵Te, ¹²⁶Te, ¹²⁸Te, ¹³⁰Te, and combinations thereof.

61. (canceled)

62. The compound of claim 41, wherein the solvate is a DMSO solvate.

63. The compound of claim 44, wherein the solvate is a DMSO solvate.

64. The kit of claim 58, wherein the tellurophene analogue is in the form of a DMSO solvate.

65. The kit of claim 58, wherein the tellurium standard comprises a single tellurium isotope.

66. The kit of claim 58, wherein the plurality of tellurium standards each comprise a different tellurium isotope.
